(19) United States
(12) Patent Application Publication
(10) Pub. No.: US 2023/0382993 A1
(43) Pub. Date: Nov. 30, 2023

(54) METHODS AND COMPOSITIONS FOR PREVENTING OR DELAYING TYPE 1 DIABETES

(71) Applicant: Provention Bio, Inc., Red Bank, NJ (US)

(72) Inventors: Francisco Leon, Bethesda, MD (US); Eleanor L. Ramos, Pacifica, CA (US)

(73) Assignee: Provention Bio, Inc., Red Bank, NJ (US)

(21) Appl. No.: 18/321,964

(22) Filed: May 23, 2023

Related U.S. Application Data
(60) Provisional application No. 63/345,365, filed on May 24, 2022.
(60) Provisional application No. 63/367,992, filed on Jul. 8, 2022.
(60) Provisional application No. 63/382,382, filed on Nov. 4, 2022.

(51) Int. Cl. C07K 16/28; A61K 45/06; G01N 33/68; A61P 3/10

(52) U.S. Cl. CPC C07K 16/2809; A61K 45/06; G01N 33/6854; A61P 3/10; A61K 2039/545

(57) ABSTRACT

Provided herein, in one aspect, is a method of preventing or delaying the onset of clinical type 1 diabetes (T1D), comprising: administering a prophylactically effective amount of an anti-CD3 antibody to a non-diabetic subject who is at risk of T1D, wherein the prophylactically effective amount has a cumulative dose of about 10,500 μg/m² to about 14,000 μg/m².

### RELATED APPLICATIONS

This application claims the benefit of and priority to U.S. Provisional Application Ser. No. 63/345,365, filed May 24, 2022, U.S. Provisional Application Ser. No. 63/367,992, filed Jul. 8, 2022, and U.S. Provisional Application Ser. No. 63/382,382, filed Nov. 4, 2022, the entire contents of each of which are hereby incorporated by reference in their entireties.

### SEQUENCE LISTING

This specification includes a sequence listing submitted herewith, which includes the file entitled 178833-011703.xml having the following size: 4,136 bytes which was created May 22, 2023, the contents of which are incorporated by reference herein.

### FIELD

The present disclosure relates in general to compositions and methods of preventing or delaying the onset of clinical type 1 diabetes (T1D) in subjects at risk, and more particularly the use of anti-CD3 antibodies.

### BACKGROUND

Type 1 diabetes (T1D) is caused by the autoimmune destruction of insulin producing beta cells in the islets of Langerhans leading to dependence on exogenous insulin injections for survival. Approximately 1.6 million Americans have Type 1 diabetes, and after asthma, it remains one of the most common diseases of childhood. Despite improvements in care, most affected individuals with T1D are not able to consistently achieve desired glycemic targets. For individuals with type 1 diabetes, there are persisting concerns for increased risk of both morbidity and mortality. Two recent studies noted loss of 17.7 life-years for children diagnosed before age 10, and 11 and 13 life-years lost for adult-diagnosed Scottish men and women respectively.

In genetically susceptible individuals, T1D progresses through asymptomatic stages prior to overt hyperglycemia, characterized first by the appearance of autoantibodies (Stage 1) and then dysglycemia (Stage 2). In Stage 2, metabolic responses to a glucose load are impaired but other metabolic indices, for example glycosylated hemoglobin, are normal and insulin treatment is not needed. These immunologic and metabolic features identify individuals who are at high-risk for development of clinical disease with overt hyperglycemia and requirement for insulin treatment (Stage 3). Several immune interventions have been shown to delay decline in beta cell function when studied in recent-onset clinical T1D. One promising therapy is the FcR non-binding anti-CD3 monoclonal antibody teplizumab, as several studies have shown that short-term treatment reduces loss of βcell function durably, with an observable effect seen as long as 7 years after diagnosis and treatment. The drug modifies the function of CD8+ T lymphocytes, which are thought to be important effector cells that cause beta cell killing.

To date, no intervention initiated before the clinical diagnosis (i.e., at Stage 1 or 2) has altered progression to clinical, Stage 3 T1D. Thus, a need exists for a treatment that would prevent or delay the onset of clinical T1D in high-risk individuals.

### SUMMARY

Aspects of the disclosure relate to a method of preventing or delaying onset of clinical type 1 diabetes (T1D), comprising: administering a prophylactically effective amount of an anti-CD3 antibody to a non-diabetic subject who is at risk for T1D.

In some embodiments, the method of preventing or delaying onset of clinical type 1 diabetes (T1D), comprises administering a prophylactically effective amount of an anti-CD3 antibody to a non-diabetic subject who is at risk of T1D, wherein the prophylactically effective amount has a cumulative dose of from about 10,500 μg/m² to about 14,000 μg/m².

In some embodiments, the non-diabetic subject is an adult or a pediatric subject. In some embodiments, the pediatric subject is 8 years old or older. In some embodiments, the pediatric subject is 7 years old or older. In some embodiments, the pediatric subject is 6 years old or older. In some embodiments, the pediatric subject is 5 years old or older. In some embodiments, the pediatric subject is 4 years old or older. In some embodiments, the pediatric subject is 3 years old or older. In some embodiments, the pediatric subject is 3 years old or older. In some embodiments, the pediatric subject is 2 year old or older. In some embodiments, the pediatric subject is 1 year old or older. In some embodiments, the pediatric subject is 1 year old or younger. In some embodiments, the pediatric subject is an infant.

In some embodiments, the non-diabetic subject is a relative of a patient with T1D.

In some embodiments, the method further includes determining that the non-diabetic subject (1) is negative for zinc transporter 8 (ZnT8) antibodies, (2) is HLA-DR4+, and/or (3) is not HLA-DR3+.

In some embodiments, the non-diabetic subject has 2 or more diabetes-related autoantibodies selected from islet cell antibodies (ICA), insulin autoantibodies (IAA), and antibodies to glutamic acid decarboxylase (GAD), tyrosine phosphatase (IA-2/ICA512) or ZnT8.

In some embodiments, the non-diabetic subject has abnormal glucose tolerance on oral glucose tolerance test (OGTT). In some embodiments, the abnormal glucose tolerance on OGTT is a fasting glucose level of 110-125 mg/dL, or 2 hour plasma of ≥140 and <200 mg/dL, or an intervening glucose value at 30, 60, or 90 minutes on OGTT>200 mg/dL.

In some embodiments, the non-diabetic subject has abnormal results in a glucose monitoring system (CGM) revealing high sensor average glucose levels (>=110 mg/dL), or high variability of glycemia (CV>=15), or less time in range (>=10% of the time above 140 mg/dL).

In some embodiments, the non-diabetic subject does not have antibodies against ZnT8.

In some embodiments, the non-diabetic subject is HLA-DR4+ and is not HLA-DR3+.

In some embodiments, the non-diabetic subject does not have antibodies against ZnT8, is HLA-DR4+ and is not HLA-DR3+.

In some embodiments, the non-diabetic subject is a relative of a patient with T1D and does not have antibodies against ZnT8. In some embodiments, the non-diabetic subject is a relative of a patient with T1D, does not have antibodies against ZnT8, is HLA-DR4+ and is not HLA-DR3+.

In some embodiments, the anti-CD3 antibody is selected from teplizumab, otelixizumab or foralumab. In some embodiments, the anti-CD3 antibody is teplizumab.

In some embodiments, the prophylactically effective amount of the antibody comprises a 10 to 14-day course of subcutaneous (SC) injection or intravenous (IV) infusion or oral administration of the anti-CD3 antibody at a cumulative dose greater than 10,500 micrograms/meter squared (μg/m²). In some embodiments, the prophylactically effective amount of the antibody comprises a 14-day course of subcutaneous (SC) injection or intravenous (IV) infusion or oral administration of the anti-CD3 antibody at a cumulative dose of from about 11,000 μg/m² to about 14,000 μg/m². In some embodiments, the anti-CD3 antibody is or comprises teplizumab.

In some embodiments, the prophylactically effective amount of the antibody comprises a 10 to 14 day course of subcutaneous (SC) injection or intravenous (IV) infusion or oral administration of the anti-CD3 antibody at from about 10,500 to about 14,000 μg/m², from about 10,500 to about 13,500 μg/m², from about 10,500 to about 13,000 μg/m², from about 10,500 to about 12,500 μg/m², from about 10,500 to about 12,000 μg/m², from about 10,500 to about 11,500 μg/m², or from about 10,500 to about 11,000 μg/m². In some embodiments, the prophylactically effective amount of the antibody comprises a 10 to 14-day course of subcutaneous (SC) injection or intravenous (IV) infusion or oral administration of the anti-CD3 antibody at about 10,500 μg/m², 11,000 μg/m², 11,500 μg/m², 12,000 μg/m², 12,500 μg/m², 13,000 μg/m², 13,500 μg/m², or 14,000 μg/m². In some embodiments, the anti-CD3 antibody is or comprises teplizumab.

In some embodiments, the method comprises administering to the non-diabetic subject who is at risk for T1D a 14-day course IV infusion of the anti-CD3 antibody at about 60 μg/m², about 125 μg/m², about 250 μg/m², and about 500 μg/m², on days 1-4, respectively, and a dose of about 1,000 μg/m² on each of days 5-14. In some embodiments, the cumulative dose is about 10,935 μg/m². In some embodiments, the anti-CD3 antibody is selected from teplizumab, otelixizumab or foralumab. In some embodiments, the anti-CD3 antibody is teplizumab. In some embodiments, the method comprises administering to the non-diabetic subject who is at risk for T1D a 14-day course IV infusion of teplizumab at about 60 μg/m², about 125 μg/m², about 250 μg/m², and about 500 μg/m², on days 1-4, respectively, and a dose of about 1,000 μg/m² on each of days 5-14.

In some embodiments, the method comprises administering to the non-diabetic subject who is at risk for T1D a 14-day course IV infusion of the anti-CD3 antibody at about 60 μg/m², about 125 μg/m², about 250 μg/m², and about 500 μg/m², on days 1-4, respectively, and a dose of about 1,030 μg/m² on each of days 5-14. In some embodiments, the cumulative dose is about 11,235 μg/m². In some embodiments, the anti-CD3 antibody is selected from teplizumab, otelixizumab or foralumab. In some embodiments, the anti-CD3 antibody is teplizumab. In some embodiments, the method comprises administering to the non-diabetic subject who is at risk for T1D a 14-day course IV infusion of teplizumab at about 60 μg/m², about 125 μg/m², about 250 μg/m², and about 500 μg/m², on days 1-4, respectively, and a dose of about 1,030 μg/m² on each of days 5-14.

In some embodiments, the method comprises administering to the non-diabetic subject who is at risk for T1D a 14-day course IV infusion of the anti-CD3 antibody at about 100 μg/m², about 425 μg/m², about 850 μg/m², and about 850 μg/m², on days 1-4, respectively, and a dose of about 1,000 μg/m² on each of days 5-14. In some embodiments, the cumulative dose is about 12,225 μg/m². In some embodiments, the anti-CD3 antibody is selected from teplizumab, otelixizumab or foralumab. In some embodiments, the anti-CD3 antibody is teplizumab. In some embodiments, the method comprises administering to the non-diabetic subject who is at risk for T1D a 14-day course IV infusion of teplizumab at about 100 μg/m², about 425 μg/m², about 850 μg/m², and about 850 μg/m², on days 1-4, respectively, and a dose of about 1,000 μg/m² on each of days 5-14.

In some embodiments, the method comprises administering to the non-diabetic subject who is at risk for T1D a 14-day course IV infusion of the anti-CD3 antibody at about 65 μg/m², about 125 μg/m², about 250 μg/m², and about 500 μg/m², on days 1-4, respectively, and a dose of about 1,070 μg/m² on each of days 5-14. In some embodiments, the cumulative dose is about 11,640 μg/m². In some embodiments, the anti-CD3 antibody is selected from teplizumab, otelixizumab or foralumab. In some embodiments, the anti-CD3 antibody is teplizumab. In some embodiments, the method comprises administering to the non-diabetic subject who is at risk for T1D a 14-day course IV infusion of teplizumab at about 65 μg/m², about 125 μg/m², about 250 μg/m², and about 500 μg/m², on days 1-4, respectively, and a dose of about 1,070 μg/m² on each of days 5-14.

In some embodiments, the method comprises administering to the non-diabetic subject who is at risk for T1D a 14-day course IV infusion of the anti-CD3 antibody at about 65 μg/m², about 125 μg/m², about 250 μg/m², and about 500 μg/m², on days 1-4, respectively, and a dose of about 1,030 μg/m² on each of days 5-14. In some embodiments, the cumulative dose is about 11,240 μg/m². In some embodiments, the anti-CD3 antibody is selected from teplizumab, otelixizumab or foralumab. In some embodiments, the anti-CD3 antibody is teplizumab. In some embodiments, the method comprises administering to the non-diabetic subject who is at risk for T1D a 14-day course IV infusion of teplizumab at about 65 μg/m², about 125 μg/m², about 250 μg/m², and about 500 μg/m², on days 1-4, respectively, and a dose of about 1,030 μg/m² on each of days 5-14.

In some embodiments, the prophylactically effective amount delays median time to clinical diagnosis of T1D by at least 50%, at least 80%, or at least 90%. In some embodiments, the prophylactically effective amount delays median time to clinical diagnosis of T1D by at least 12 months, at least 18 months, at least 24 months, at least 36 months, at least 48 months, or at least 60 months.

In some embodiments, the method further comprises determining, prior to or after the administering step, that the non-diabetic subject has more than about 10% TIGIT+KLRG1+CD8+ T-cells in all CD3+ T cells, which is indicative of successful prevention or delay of the onset of clinical T1D. In some embodiments, the step of determining of TIGIT+KLRG1+CD8+ T-cells is by flow cytometry. In some embodiments, the method further comprises determining a decrease in a percentage of CD8+ T cells expressing proliferation markers Ki67 and/or CD57.

Some aspects of the disclosure relate to a method of preventing or delaying onset of clinical type 1 diabetes (T1D), comprising: administering to a non-diabetic subject 8 years of age or older who is at risk of T1D a 14 day course IV infusion of teplizumab at about 65 μg/m² on day 1, about 125 μg/m² on day 2, about 250 μg/m² on day 3, and about 500 μg/m² on day 4, and a dose of about 1,030 μg/m² on each of days 5-14.

Some aspects of the disclosure relate to a method of delaying onset of Stage 3 type 1 diabetes (T1D), comprising administering to a subject in need thereof who has a diagnosis of stage 2 T1D a 14 day course IV infusion of teplizumab at about 65 μg/m² on day 1, about 125 μg/m² on day 2, about 250 μg/m² on day 3, and about 500 μg/m² on day 4, and a dose of about 1,030 μg/m² on each of days 5-14. In some embodiments, the non-diabetic subject is an adult or a pediatric subject. In some embodiments, the pediatric subject is 8 years old or older. In some embodiments, the pediatric subject is 7 years old or older. In some embodiments, the pediatric subject is 6 years old or older. In some embodiments, the pediatric subject is 5 years old or older. In some embodiments, the pediatric subject is 4 years old or older. In some embodiments, the pediatric subject is 3 years old or older. In some embodiments, the pediatric subject is 3 years old or older. In some embodiments, the pediatric subject is 2 year old or older. In some embodiments, the pediatric subject is 1 year old or older. In some embodiments, the pediatric subject is 1 year old or younger. In some embodiments, the pediatric subject is an infant.

In some embodiments, the method comprises documenting at least two positive pancreatic islet autoantibodies in the subject who has dysglycemia without overt hyperglycemia before administering the 14 day course.

In some embodiments, the subject in need thereof has dysglycemia without overt hyperglycemia and has two or more pancreatic islet autoantibodies.

In some embodiments, the two or more pancreatic islet autoantibodies comprise islet cell antibodies (ICA), insulin autoantibodies (IAA), and antibodies to glutamic acid decarboxylase (GAD), tyrosine phosphatase (IA-2/ICA512) or ZnT8.

In some embodiments, the method comprises administering on at least each of days 1-5, and prior to the administering of the 14 days course IV infusion, an effective amount of a nonsteroidal anti-inflammatory drug (NSAID), acetaminophen, an antihistamine, an antiemetic or a combination thereof. In some embodiments, the method comprises administering orally the NSAID, acetaminophen, antihistamine, antiemetic or combination thereof.

In some embodiments, the method comprises administering the 14 days course IV infusion once daily for 14 consecutive days over a period of at least 30 minutes.

In some embodiments, the method further comprises determining, prior to or after the administering step, that the non-diabetic subject has more than about 10% TIGIT+KLRG1+CD8+ T-cells in all CD3+ T cells, which is indicative of successful prevention or delay of the onset of clinical T1D.

In some embodiments, the determining of TIGIT+KLRG1+CD8+ T-cells is by flow cytometry.

In some embodiments, the method further includes determining a decrease in a percentage of CD8+ T cells expressing proliferation markers Ki67 and/or CD57.

Other aspects of the disclosure relate to a method of prognosing responsiveness of an anti-CD3 antibody in preventing or delaying onset of type 1 diabetes (T1D), the method comprising administering a prophylactically effective amount of an anti-CD3 antibody to a non-diabetic subject who is at risk for T1D, wherein the prophylactically effective amount has a cumulative dose of about 10,500 μg/m² to about 14,000 μg/m²; and determining C-peptide area under the curve (AUC): glucose AUC ratio, wherein an increase in the ratio indicates responsiveness to the anti-CD3 antibody and/or non-progression to clinical T1D.

Aspects of the disclosure relate to an anti-CD3 antibody for use in a method of preventing or delaying onset of clinical type 1 diabetes (T1D), comprising administering a prophylactically effective amount of an anti-CD3 antibody to a non-diabetic subject who is at risk of T1D, wherein the prophylactically effective amount has a cumulative dose of about 10,500 μg/m² to about 14,000 μg/m². In some embodiments, the anti-CD3 antibody is selected from teplizumab, otelixizumab or foralumab. In some embodiments, the anti-CD3 antibody is teplizumab.

Aspects of the disclosure relate to a method of preventing or delaying onset of Stage 3 type 1 diabetes (T1D), comprising administering to a subject who has a diagnosis of stage 2 T1D a 14 day course IV infusion of teplizumab at about 65 μg/m² on day 1, about 125 μg/m² on day 2, about 250 μg/m² on day 3, and about 500 μg/m² on day 4, and a dose of about 1,030 μg/m² on each of days 5-14.

In some embodiments, the subject in need thereof has two or more pancreatic islet autoantibodies. In some embodiments, the subject in need thereof has dysglycemia without overt hyperglycemia and has two or more pancreatic islet autoantibodies. In some embodiments, the method comprises confirming stage 2 T1D by documenting at least two positive pancreatic islet autoantibodies in subjects who have dysglycemia without overt hyperglycemia before administering the 14 day course. In some embodiments, the pancreatic islet autoantibodies comprise islet cell antibodies (ICA), insulin autoantibodies (IAA), antibodies to glutamic acid decarboxylase (GAD), tyrosine phosphatase (IA-2/ICA512) or ZnT8.

### BRIEF DESCRIPTION OF THE DRAWINGS

FIG. 1 shows AUC_{inf}, Cₘₐₓ and C_{trough13}: Reference Regimen and Simulated Alternative Regimens (Regimen A, Regimen B, regimen C). Exposures for the reference regimen reflect the Herold 14-day regimen administered to PROTECT study participants who received the anti-CD3 antibody. Exposures for alternative regimens reflect simulated anti-CD3 antibody administered to a similar population of T1D patients as a subset of PROTECT study participants. The boxes indicate the median (line within a box) values as well as Q1 to Q3 interquartile ranges (IQRs). The whiskers indicate Q1−1.5 IQR and Q3+1.5 IQR. The data points outside the whiskers indicate outlier values.

FIG. 2 shows AUC_{inf}, Cₘₐₓ and C_{trough13}: Historical Clinical Trials and Simulated Alternative Regimens for the anti-CD3 antibody. Protégé and TN-10 exposures represent all patients who received the Herold 14-day regimen in these studies; PROTECT exposures represent patients who received the anti-CD3 antibody in the PROTECT study. Regimen A, Regimen B and Regimen C graphs are simulated PK data for anti-CD3 antibody Regimen A, Regimen B and Regimen C using in a similar population of T1D patients as a subset of PROTECT study subjects. The boxes indicate the median (line within a box) values as well as Q1 to Q3 interquartile ranges (IQRs). The whiskers indicate Q1−1.5 IQR and Q3+1.5 IQR. The data points outside the whiskers indicate outlier values.

FIG. 3 shows Predicted Cₘₐₓ and C_{trough} Levels During 14-day Treatment Course: Regimens A and B. The boxes indicate the median (line within a box) values as well as Q1 to Q3 interquartile ranges (IQRs). The whiskers indicate Q1−1.5 IQR and Q3+1.5 IQR. The data points outside the whiskers indicate outlier values.

FIG. 4 shows Predicted Cₘₐₓ and C_{trough} Levels During 14-day Treatment Course: Regimen C. The boxes indicate the median (line within a box) values as well as Q1 to Q3 interquartile ranges (IQRs). The whiskers indicate Q1−1.5 IQR and Q3+1.5 IQR. The data points outside the whiskers indicate outlier values.

FIG. 5 shows AUC_{inf}, Cₘₐₓ and C_{trough13}: Reference Regimen and Simulated Regimen D. Exposures for the reference regimen reflect the Herold 14-day regimen administered to PROTECT study participants who received the anti-CD3 antibody. Exposures for alternative regimens reflect simulated anti-CD3 antibody administered to a similar population of T1D patients as a subset of PROTECT study participants. The boxes indicate the median (line within a box) values as well as Q1 to Q3 interquartile ranges (IQRs). The whiskers indicate Q1−1.5 IQR and Q3+1.5 IQR. The data points outside the whiskers indicate outlier values.

FIG. 6 shows AUC_{inf}, Cₘₐₓ and C_{trough13}: Historical Clinical Trials and Simulated Regimen D for the anti-CD3 antibody. Protégé and TN-10 exposures represent all patients who received the Herold 14-day regimen in these studies; PROTECT exposures represent patients who received the anti-CD3 antibody in the PROTECT study. Regimen D is simulated PK data for anti-CD3 antibody Regimen D using in a similar population of T1D patients as a subset of PROTECT study subjects. The boxes indicate the median (line within a box) values as well as Q1 to Q3 interquartile ranges (IQRs). The whiskers indicate Q1−1.5 IQR and Q3+1.5 IQR. The data points outside the whiskers indicate outlier values.

FIG. 7 shows Predicted Cₘₐₓ and C_{trough} Levels During 14-day Treatment Course: Regimen D. The boxes indicate the median (line within a box) values as well as Q1 to Q3 interquartile ranges (IQRs). The whiskers indicate Q1−1.5 IQR and Q3+1.5 IQR. The data points outside the whiskers indicate outlier values.

### DETAILED DESCRIPTION

Provided herein, in some embodiments, is a method of preventing or delaying the onset of clinical type 1 diabetes (T1D), comprising: providing a non-diabetic subject who is at risk for T1D; administering a prophylactically effective amount of an anti-CD3 antibody to the non-diabetic subject; and determining, prior to or after the administering step, that the non-diabetic subject has more than about 10% TIGIT+KLRG1+CD8+ T-cells in all CD3+ T cells, which is indicative of successful prevention or delay of the onset of clinical T1D.

In some embodiments, a method of prognosing responsiveness of an anti-CD3 antibody, e.g., teplizumab, in preventing or delaying the onset of T1D is provided. The method can include: providing a non-diabetic subject who is at risk for T1D; administering a prophylactically effective amount of the anti-CD3 antibody, e.g., teplizumab, to the non-diabetic subject; and determining C-peptide area under the curve (AUC): glucose AUC ratio, wherein an increase in the ratio indicates responsiveness to the anti-CD3 antibody.

### Definitions

Certain terms are defined herein below. Additional definitions are provided throughout the application.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 30 percent (%), within 25% within 20%, for example, within 10%, or, within 5% of a given range of values. The term “substantially” means more than 50%, more than 60%, more than 70% more than 80%, and more than 90% or 95%.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

As used herein, the term “prophylactic agent” refers to CD3 binding molecules such as teplizumab which can be used in the prevention, treatment, management or amelioration of one or more symptoms of T1D.

As used herein, the term “onset” of disease with reference to Type-1 diabetes refers to a patient meeting the criteria established for diagnosis of Type-1 diabetes by the American Diabetes Association (see, Mayfield et al., 2006, Am. Fam. Physician 58:1355-1362).

As used herein, the terms “prevent”, “preventing” and “prevention” refer to the prevention of the onset of one or more symptoms of T1D in a subject resulting from the administration of a prophylactic or therapeutic agent.

As used herein, a “protocol” includes dosing schedules and dosing regimens. The protocols herein are methods of use and include prophylactic and therapeutic protocols. A “dosing regimen” or “course of treatment” may include administration of several doses of a therapeutic or prophylactic agent over 1 to 20 days.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey or a human), and more preferably a human.

As used herein, the term “prophylactically effective amount” refers to that amount of teplizumab sufficient to result in the delay or prevention of the development, recurrence or onset of one or more symptoms of T1D. In some embodiments, a prophylactically effective amount preferably refers to the amount of teplizumab that delays a subject's onset of T1D by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%.

Various aspects of the disclosure are described in further detail below. Additional definitions are set out throughout the specification.

### Anti-CD3 Antibodies and Pharmaceutical Compositions

The terms “anti-CD3 antibody” and “an antibody that binds to CD3” refer to an antibody or antibody fragment that is capable of binding cluster of differentiation 3 (CD3) with sufficient affinity such that the antibody is useful as a prophylactic, diagnostic and/or therapeutic agent in targeting CD3. In some embodiments, the extent of binding of an anti-CD3 antibody to an unrelated, non-CD3 protein is less than about 10% of the binding of the antibody to CD3 as measured, e.g., by a radioimmunoassay (RIA). In some embodiments, an antibody that binds to CD3 has a dissociation constant (Kd) of <1 μM, <100 nM, <10 nM, <1 nM, <0.1 nM, <0.01 nM, or <0.001 nM (e.g. 10⁻⁸ M or less, e.g. from 10⁻⁸ M to 10⁻¹³ M, e.g., from 10⁻⁹ M to 10⁻¹³ M). In some embodiments, an anti-CD3 antibody binds to an epitope of CD3 that is conserved among CD3 from different species.

In some embodiments, the anti-CD3 antibody can be ChAglyCD3 (otelixizumab). Otelixizumab is a humanized Fc nonbinding anti-CD3, which was evaluated initially in phase 2 studies by the Belgian Diabetes Registry (BDR) and then developed by Tolerx, which then partnered with GSK to conduct the phase 3 DEFEND new onset T1D trials (NCT00678886, NCT01123083, NCT00763451). Otelixizumab is administered IV with infusions over 8 days. See, e.g., Wiczling et al., J. Clin. Pharmacol. 50 (5) (May 2010) 494-506; Keymeulen et al., N Engl J Med. 2005; 352:2598-608; Keymeulen et al., Diabetologia. 2010; 53:614-23; Hagopian et al., Diabetes. 2013; 62:3901-8; Aronson et al., Diabetes Care. 2014; 37:2746-54; Ambery et al., Diabet Med. 2014; 31:399-402; Bolt et al., Eur. J. Immunol. lYY3. 23: 403-411; Vlasakakis et al., Br J Clin Pharmacol (2019) 85 704-714; Guglielmi et al, Expert Opinion on Biological Therapy, 16:6, 841-846; Keymeulen et al., N Engl J Med 2005; 352:2598-608; Keymeulen et al., BLOOD 2010, VOL 115, No. 6; Sprangers et al., Immunotherapy (2011) 3 (11), 1303-1316; Daifotis et al., Clinical Immunology (2013) 149, 268-278; all incorporated herein by reference.

In some embodiments, the anti-CD3 antibody can be visilizumab (also called HuM291; Nuvion). Visilizumab is a humanized anti-CD3 monoclonal antibody characterized by a mutated IgG2 isotype, lack of binding to Fcγ receptors, and the ability to induce apoptosis selectively in activated T cells. It was evaluated in patients in graft-versus-host disease (NCT00720629; NCT00032279) and in ulcerative colitis (NCT00267306) and Crohn's Disease (NCT00267709). See, e.g., Sandborn et al., Gut 59 (11) (November 2010) 1485-1492, incorporated herein by reference.

In some embodiments, the anti-CD3 antibody can be foralumab, a fully human anti-CD3 monoclonal antibody being developed by Tiziana Life Sciences, PLC in NASH and T2D (NCT03291249). See, e.g., Ogura et al., Clin Immunol. 2017; 183:240-246; Ishikawa et al., Diabetes. 2007; 56 (8):2103-9; Wu et al., J Immunol. 2010; 185 (6):3401-7; all incorporated herein by reference.

In some embodiments, the anti-CD3 antibody can be teplizumab. Teplizumab, also known as hOKT3y1(Ala-Ala) (containing an alanine at positions 234 and 235) is an anti-CD3 antibody that had been engineered to alter the function of the T lymphocytes that mediate the destruction of the insulin-producing beta cells of the islets of the pancreas. Teplizumab binds to an epitope of the CD3ε chain expressed on mature T cells and by doing so changes their function. Sequences and compositions of teplizumab are disclosed in U.S. Pat. Nos. 6,491,916; 8,663,634; and 9,056,906, each incorporated herein by reference in its entirety. The full sequences of light and heavy chains are set forth below. Bolded portions are the complementarity determining regions.

Teplizumab Light Chain (SEQ ID NO: 1):
DIQMTQSPSSLSASVGDRVTITCSASSSVSYMNWYQQTPGKAPKRWIYD
TSKLASGVPSRFSGSGSGTDYTFTISSLQPEDIATYYCQQWSSNPFTFG
QGTKLQITRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC
Teplizumab Heavy Chain (SEQ ID NO: 2):
QVQLVQSGGGVVQPGRSLRLSCKASGYTFTRYTMHWVRQAPGKGLEWIG
YINPSRGYTNYNQKVKDRFTISRDNSKNTAFLQMDSLRPEDTGVYFCAR
YYDDHYCLDYWGQGTPVTVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKKVEPKSCDKTHTCPPCPAPEAAGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQK
SLSLSPGK

In some embodiments, provided herein, is a pharmaceutical composition. Such compositions comprise a prophylactically effective amount of an anti-CD3 antibody, and a pharmaceutically acceptable carrier. In some embodiments, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like (See, for example, Handbook of Pharmaceutical Excipients, Arthur H. Kibbe (ed., 2000, which is incorporated by reference herein in its entirety), Am. Pharmaceutical Association, Washington, D.C.

The composition, if desired, can also contain minor amounts of wetting or emulsifying agents, or pH buffering agents. These compositions can take the form of solutions, suspensions, emulsion, tablets, pills, capsules, powders, sustained release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of a prophylactic or therapeutic agent preferably in purified form, together with a suitable amount of carrier so as to provide the form for proper administration to the patient. The formulation should suit the mode of administration. In some embodiments, the pharmaceutical compositions are sterile and in suitable form for administration to a subject, preferably an animal subject, more preferably a mammalian subject, and most preferably a human subject.

In some embodiments, it may be desirable to administer the pharmaceutical compositions locally to the area in need of treatment; this may be achieved by, for example, and not by way of limitation, local infusion, by injection, or by means of an implant, the implant being of a porous, non-porous, or gelatinous material, including membranes, such as sialastic membranes, or fibers. Preferably, when administering the anti-CD3 antibody, care must be taken to use materials to which the anti-CD3 antibody does not absorb.

In some embodiments, the composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In some embodiments, the composition can be delivered in a controlled release or sustained release system. In some embodiments, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In some embodiments, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In some embodiments, the polymer used in a sustained release formulation is inert, free of leachable impurities, stable on storage, sterile, and biodegradable. In some embodiments, a controlled or sustained release system can be placed in proximity of the therapeutic target, i.e., the lungs, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more antibodies of the invention or fragments thereof. See, e.g., U.S. Pat. No. 4,526,938; PCT Publication No. WO 91/05548; PCT Publication No. WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Control Rel. Bioact. Mater. 24:759-760, each of which is incorporated herein by reference in its entirety.

A pharmaceutical composition can be formulated to be compatible with its intended route of administration. Examples of routes of administration include, but are not limited to, parenteral, e.g., intravenous, intradermal, subcutaneous, oral, intranasal (e.g., inhalation), transdermal (topical), transmucosal, and rectal administration. In some embodiments, the composition is formulated in accordance with routine procedures as a pharmaceutical composition adapted for intravenous, subcutaneous, intramuscular, oral, intranasal or topical administration to human beings. In some embodiments, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

The compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. In some embodiments, the disclosure provides dosage forms that permit administration of the anti-CD3 antibody continuously over a period of hours or days (e.g., associated with a pump or other device for such delivery), for example, over a period of 1 hour, 2 hours, 3 hours, 4 hours, 6 hours, 8 hours, 10 hours, 12 hours, 16 hours, 20 hours, 24 hours, 30 hours, 36 hours, 4 days, 5 days, 7 days, 10 days or 14 days. In some embodiments, the invention provides dosage forms that permit administration of a continuously increasing dose, for example, increasing from 51 ug/m²/day to 826 ug/m²/day over a period of 24 hours, 30 hours, 36 hours, 4 days, 5 days, 7 days, 10 days or 14 days.

The compositions can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of the compositions disclosed herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle or bag containing sterile pharmaceutical grade water or saline. In some embodiments, the composition is in solution. In some embodiments, the composition is packaged into a vial (e.g. sterile glass vial of 0.9% sodium chloride injection) or into an infusion bag (e.g. Polyvinylchloride (PVC) infusion bag of 0.9% sodium chloride injection). In some embodiments, the vial is for single-dose. Where the composition is administered by injection, an ampoule of sterile water for injection or saline can be provided so that the ingredients may be mixed prior to administration.

In particular, the disclosure provides that the anti-CD3 antibodies, or pharmaceutical compositions thereof, can be packaged in a hermetically sealed container such as an ampoule or sachette indicating the quantity of the agent. In some embodiments, the anti-CD3 antibody, or pharmaceutical compositions thereof is supplied as a dry sterilized lyophilized powder or water free concentrate in a hermetically sealed container and can be reconstituted, e.g., with water or saline to the appropriate concentration for administration to a subject. Preferably, the anti-CD3 antibody, or pharmaceutical compositions thereof is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic agents, or pharmaceutical compositions herein should be stored at between 2° C. and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In some embodiments, the pharmaceutical composition is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/ml, at least 25 mg/ml, at least 50 mg/ml, at least 75 mg/ml or at least 100 mg/ml. The liquid form should be stored at between 2° C. and 8° C. in its original container.

In some embodiments, the disclosure provides that the composition of the invention is packaged in a hermetically sealed container such as an ampoule or sachette indicating the quantity of the anti-CD3 antibody.

The compositions may, if desired, be presented in a pack or dispenser device that may contain one or more unit dosage forms containing the active ingredient. The pack may, for example, comprise metal or plastic foil, such as a blister pack.

The amount of the composition of the invention which will be effective in the prevention or amelioration of one or more symptoms associated with T1D can be determined by standard clinical techniques. The precise dose to be employed in the formulation will also depend on the route of administration and the seriousness of the condition, and should be decided according to the judgment of the practitioner and each patient's circumstances. Effective doses may be extrapolated from dose-response curves derived from in vitro or animal model test systems.

### Methods and Use

In some embodiments, the present disclosure encompasses administration of anti-human CD3 antibodies such as teplizumab to individuals predisposed to develop type 1 diabetes or with pre-clinical stages of type 1 diabetes, but who do not meet the diagnosis criteria as established by the American Diabetes Association or the Immunology of Diabetes Society to prevent or delay the onset of type 1 diabetes and/or to prevent or delay the need for administration of insulin to such patients. In some embodiments, high-risk factors for identification of predisposed subjects include having first or second degree relatives with diagnosed type-1 diabetes, an impaired fasting glucose level (e.g., at least one determination of a glucose level of 100-125 mg/dl after fasting (8 hours with no food)), an impaired glucose tolerance in response to a 75 g OGTT (e.g., at least one determination of a 2-hr glucose level of 140-199 mg/dl in response to a 75 g OGTT), an HLA type of DR3, DR4 or DR7 in a Caucasian, an HLA type of DR3 or DR4 in a person of African descent, an HLA type of DR3, DR4 or DR9 in a person of Japanese descent, exposure to viruses (e.g., coxsackie B virus, enteroviruses, adenoviruses, rubella, cytomegalovirus, Epstein-Barr virus), a positive diagnosis according to art accepted criteria of at least one other autoimmune disorder (e.g., thyroid disease, celiac disease), and/or the detection of autoantibodies, particularly ICAs and type 1 diabetes-associated autoantibodies, in the serum or other tissues. In some embodiments, the subject identified as predisposed to developing type 1 diabetes has at least one of the risk factors described herein and/or as known in the art. The present disclosure also encompasses identification of subjects predisposed to development of type 1 diabetes, wherein the subject presents a combination of two or more, three or more, four or more, or more than five of the risk factors disclosed herein or known in the art. In some embodiments, the non-diabetic subject is an adult or a pediatric subject. In some embodiments, the pediatric subject is 8 years old or older. In some embodiments, the pediatric subject is 7 years old or older. In some embodiments, the pediatric subject is 6 years old or older. In some embodiments, the pediatric subject is 5 years old or older. In some embodiments, the pediatric subject is 4 years old or older. In some embodiments, the pediatric subject is 3 years old or older. In some embodiments, the pediatric subject is 2 years old or older. In some embodiments, the pediatric subject is 1 year old or older. In some embodiments, the pediatric subject is an infant. In some embodiments, the infant is 12 months old or less, 11 months old or less, 10 months old of less, 9 months old or less, 8 months old or less, 7 months old or less, 6 months old or less, 5 months old or less, 4 months old or less, 3 months old or less, 2 months old or less, 1 month old or less.

Serum autoantibodies associated with type 1 diabetes or with a predisposition for the development of type 1 diabetes are islet-cell autoantibodies (e.g., anti-ICA512 autoantibodies), glutamic acid decarbamylase autoantibodies (e.g., anti-GAD65 autoantibodies), IA2 antibodies, ZnT8 antibodies and/or anti-insulin autoantibodies. Accordingly, in a specific example in accordance with this embodiment, the invention encompasses the treatment of an individual with detectable autoantibodies associated with a predisposition to the development of type 1 diabetes or associated with early stage type 1 diabetes (e.g., anti-IA2, anti-ICA512, anti-GAD or anti-insulin autoantibodies), wherein the individual has not been diagnosed with type 1 diabetes and/or is a first or second degree relative of a type-1 diabetic. In some embodiments, the presence of the autoantibodies is detected by ELISA, electrochemoluminescence (ECL), radioassay (see, e.g., Yu et al., 1996, J. Clin. Endocrinol. Metab. 81:4264-4267), agglutination PCR (Tsai et al, ACS Central Science 2016 2 (3), 139-147) or by any other method for immunospecific detection of antibodies described herein or as known to one of ordinary skill in the art.

β-cell function prior to, during, and after therapy may be assessed by methods described herein or by any method known to one of ordinary skill in the art. For example, the Diabetes Control and Complications Trial (DCCT) research group has established the monitoring of percentage glycosylated hemoglobin (HA1 and HA1c) as the standard for evaluation of blood glucose control (DCCT, 1993, N. Engl. J. Med. 329:977-986). Alternatively, characterization of daily insulin needs, C-peptide levels/response, hypoglycemic episodes, and/or FPIR may be used as markers of β-cell function or to establish a therapeutic index (See Keymeulen et al., 2005, N. Engl. J. Med. 352:2598-2608; Herold et al., 2005, Diabetes 54:1763-1769; U.S. Pat. Appl. Pub. No. 2004/0038867 A1; and Greenbaum et al., 2001, Diabetes 50:470-476, respectively). For example, FPIR is calculated as the sum of insulin values at 1 and 3 minutes post IGTT, which are performed according to Islet Cell Antibody Register User's Study protocols (see, e.g., Bingley et al., 1996, Diabetes 45:1720-1728 and McCulloch et al., 1993, Diabetes Care 16:911-915).

In some embodiments, the individuals predisposed to develop T1D can be a non-diabetic subject who is a relative of a patient with T1D. In some embodiments, the non-diabetic subject has 2 or more diabetes-related autoantibodies selected from islet cell antibodies (ICA), insulin autoantibodies (IAA), and antibodies to glutamic acid decarboxylase (GAD), tyrosine phosphatase (IA-2/ICA512) or ZnT8.

In some embodiments, the non-diabetic subject has abnormal glucose tolerance on oral glucose tolerance test (OGTT). Abnormal glucose tolerance on OGTT is defined as a fasting glucose level of 110-125 mg/dL, or 2 hour plasma of ≥140 and <200 mg/dL, or an intervening glucose value at 30, 60, or 90 minutes on OGTT>200 mg/dL.

In some embodiments, the non-diabetic subject has abnormal results in a glucose monitoring system (CGM) revealing high sensor average glucose levels (>=110 mg/dL), or high variability of glycemia (CV>=15), or less time in range (>=10% of the time above 140 mg/dL).

In some embodiments, the non-diabetic subject who will respond to the anti-CD3 antibody such as teplizumab does not have antibodies against ZnT8. In some embodiments, such non-diabetic subject is HLA-DR4+ and is not HLA-DR3+. In some embodiments, such non-diabetic subject who will respond to the anti-CD3 antibody such as teplizumab demonstrates an increase, following administration (e.g., after 1 month, after 2 months, after 3 months, or longer or shorter), in the frequency (or relative amount) of TIGIT+KLRG1+CD8+ T-cells (e.g., by flow cytometry) in peripheral blood mononuclear cells.

In some embodiments, the methods provided herein comprise administering an anti-human CD3 antibodies such as teplizumab to a subject in need thereof with Stage 2 type 1 diabetes. In some embodiments, the subject is an adult. In some embodiments, the subject is a pediatric subject. In some embodiments, the pediatric subject is 8 years old or older (e.g. 8 years old, 9 years old, 10 years old, 11 years old, 12 years old, 13 years old, 14 years old, 15 years old, 16 years old, 17 years old, 18 years old or older). In some embodiments, administration of the anti-human CD3 antibodies such as teplizumab delay the onset of Stage 3 Type 1 diabetes. In some embodiments, the subject has Stage 2 type 1 diabetes. In some embodiments, the subject that has dysglycemia without overt hyperglycemia has at least two positive pancreatic islet cell autoantibodies. See ADA and ISPAD guidelines which define classification and diagnosis of Stage 2 T1D in American Diabetes Association Professional Practice Committee—Classification and Diagnosis of Diabetes: Standards of Medical Care in Diabetes-2022. Diabetes Care. 2022 Jan. 1; 45 (Suppl 1):517-538); and in Besser R E J, Bell K J, Couper J J, Ziegler A G, Wherrett D K, Knip M, Speake C, Casteels K, Driscoll K A, Jacobsen L, Craig M E, Haller M J. ISPAD clinical practice consensus guidelines 2022: Stages of type 1 diabetes in children and adolescents. Pediatr Diabetes. 2022 Sep. 30). In some embodiments, the subject has abnormal glucose tolerance on oral glucose tolerance test (OGTT). In some embodiments, the subject in need thereof meets criteria for a diagnosis of Stage 2 type 1 diabetes does not have type 2 diabetes. In some embodiments, the methods provided herein comprise confirming that the subject who has dysglycemia without overt hyperglycemia has Stage 2 type 1 diabetes by documenting at least two positive pancreatic islet cell autoantibodies. In some embodiments, the methods provided herein further comprise confirming that the subject does not have type 2 diabetes. In some embodiments, subjects having Stage 2 type 1 diabetes have both of the following:

1. Two or more of the following pancreatic islet autoantibodies:
Glutamic acid decarboxylase 65 (GAD) autoantibodies
Insulin autoantibody (IAA)
Insulinoma-associated antigen 2 autoantibody (IA-2A)
Zinc transporter 8 autoantibody (ZnT8A)
Islet cell autoantibody (ICA)
   2. Dysglycemia (e.g. on oral glucose tolerance testing).

In some embodiments, Stage 2 type 1 diabetes can be diagnosed in other ways known to those skilled in the art.

Aspects of the disclosure relate to methods of preventing or delaying onset of Stage 3 type 1 diabetes (T1D). In some embodiments, the subject is an adult. In some embodiments, the subject is a child 1 year old, 2 years old, 3 years old or older, 4 years old and older, 5 years old and older, 6 years old and older, 7 years old and older, 8 years old and older, 9 years old and older, 10 years old and older, 11 years old and older, 12 years old and older, 13 years old and older, 14 years old and older, 15 years old and older, 16 years old and older, 17 years old and older, 18 years old and older. In some embodiments, the subject is a infant (12 months old or less, 11 months old or less, 10 months old or less, 9 months old or less, 8 months old or less, 7 months old or less, 6 months old or less, 5 months old or less, 4 months old or less, 3 months old or less, 2 months old or less, 1 month old or less (e.g. initial weeks of life)).

In some embodiments, the prophylactically effective amount of the antibody comprises a 10 to 14 days course of subcutaneous (SC) injection or intravenous (IV) infusion or oral administration of the anti-CD3 antibody at a cumulative dose greater than 10,000 micrograms/meter squared (μg/m²). In some embodiments, the prophylactically effective amount of the antibody comprises a 10 to 14 days course of subcutaneous (SC) injection or intravenous (IV) infusion or oral administration of the anti-CD3 antibody at from about 9,500 to about 14,000 μg/m², from about 9,500 to about 13,500 μg/m², from about 9,500 to about 13,000 μg/m², from about 9,500 to about 12,500 μg/m², from about 9,500 to about 12,000 μg/m², from about 9,500 to about 11,500 μg/m², from about 9,500 to about 11,000 μg/m², from about 9,500 to about 10,500 μg/m², from about 9,500 to about 10,000 μg/m². In some embodiments, the prophylactically effective amount of the antibody comprises a 10 to 14 days course of subcutaneous (SC) injection or intravenous (IV) infusion or oral administration of the anti-CD3 antibody at from about 10,000 to about 14,000 μg/m², from about 10,000 to about 13,500 μg/m², from about 10,000 to about 13,000 μg/m², from about 10,000 to about 12,500 μg/m², from about 10,000 to about 12,000 μg/m², from about 10,000 to about 11,500 μg/m², from about 10,000 to about 11,000 μg/m², from about 10,000 to about 10,500 μg/m². In some embodiments, the prophylactically effective amount of the antibody comprises a 10 to 14 days course of subcutaneous (SC) injection or intravenous (IV) infusion or oral administration of the anti-CD3 antibody at from about 10,500 to about 14,000 μg/m², from about 10,500 to about 13,500 μg/m², from about 10,500 to about 13,000 μg/m², from about 10,500 to about 12,500 μg/m², from about 10,500 to about 12,000 μg/m², from about 10,500 to about 11,500 μg/m², from about 10,500 to about 11,000 μg/m². In some embodiments, the prophylactically effective amount of the antibody comprises a 10 to 14 days course of subcutaneous (SC) injection or intravenous (IV) infusion or oral administration of the anti-CD3 antibody at from about 11,000 to about 14,000 μg/m², from about 11,000 to about 13,500 μg/m², from about 11,000 to about 13,000 μg/m², from about 11,000 to about 12,500 μg/m², from about 11,000 to about 12,000 μg/m², from about 11,000 to about 11,500 μg/m². In some embodiments, the prophylactically effective amount of the antibody comprises a 10 to 14 days course of subcutaneous (SC) injection or intravenous (IV) infusion or oral administration of the anti-CD3 antibody at from about 11,000 to about 14,000 μg/m², from about 11,000 to about 13,500 μg/m², from about 11,000 to about 13,000 μg/m², from about 11,000 to about 12,500 μg/m², from about 11,000 to about 12,000 μg/m², from about 11,000 to about 11,500 μg/m². In some embodiments, the prophylactically effective amount of the antibody comprises a 10 to 14 days course of subcutaneous (SC) injection or intravenous (IV) infusion or oral administration of the anti-CD3 antibody at from about 11,500 to about 14,000 μg/m², from about 11,500 to about 13,500 μg/m², from about 11,500 to about 13,000 μg/m², from about 11,500 to about 12,500 μg/m², from about 11,500 to about 12,000 μg/m². In some embodiments, the prophylactically effective amount of the antibody comprises a 10 to 14 days course of subcutaneous (SC) injection or intravenous (IV) infusion or oral administration of the anti-CD3 antibody at from about 12,000 to about 14,000 μg/m², from about 12,000 to about 13,500 μg/m², from about 12,000 to about 13,000 μg/m², from about 12,000 to about 12,500 μg/m². In some embodiments, the prophylactically effective amount of the antibody comprises a 10 to 14 days course of subcutaneous (SC) injection or intravenous (IV) infusion or oral administration of the anti-CD3 antibody at from about 12,500 to about 14,000 μg/m², from about 12,500 to about 13,500 μg/m², from about 12,500 to about 13,000 μg/m². In some embodiments, the prophylactically effective amount of the antibody comprises a 10 to 14 days course of subcutaneous (SC) injection or intravenous (IV) infusion or oral administration of the anti-CD3 antibody at from about 13,000 to about 14,000 μg/m², from about 13,000 to about 13,500 μg/m². In some embodiments, the prophylactically effective amount of the antibody comprises a 10 to 14 days course of subcutaneous (SC) injection or intravenous (IV) infusion or oral administration of the anti-CD3 antibody at from about 13,500 μg/m² to about 14,000 μg/m². In some embodiments, the prophylactically effective amount of the antibody comprises a 10 to 14 days course of subcutaneous (SC) injection or intravenous (IV) infusion or oral administration of the anti-CD3 antibody at about 10,000 μg/m², 10,500 μg/m², 11,000 μg/m², 11.500 μg/m², 12,000 μg/m², 12,500 μg/m², 13,000 μg/m², 13,500 μg/m², or 14,000 μg/m². In some embodiments, the anti-CD3 antibody is or comprises teplizumab.

In some embodiments, the prophylactically effective amount of the antibody comprises a 10 to 14 days course of intravenous (IV) infusion of teplizumab at from about 10,000 to about 14,000 μg/m², from about 10,000 to about 13,500 μg/m², from about 10,000 to about 13,000 μg/m², from about 10,000 to about 12,500 μg/m², from about 10,000 to about 12,000 μg/m², from about 10,000 to about 11,500 μg/m², from about 10,000 to about 11,000 μg/m², from about 10,000 to about 10,500 μg/m². In some embodiments, the prophylactically effective amount of the antibody comprises a 10 to 14 days course of intravenous (IV) infusion of teplizumab at from about 10,500 to about 14,000 μg/m², from about 10,500 to about 13,500 μg/m², from about 10,500 to about 13,000 μg/m², from about 10,500 to about 12,500 μg/m², from about 10,500 to about 12,000 μg/m², from about 10,500 to about 11,500 μg/m², from about 10,500 to about 11,000 μg/m². In some embodiments, the prophylactically effective amount of the antibody comprises a 10 to 14 days course of intravenous (IV) infusion of teplizumab at from about 11,000 to about 14,000 μg/m², from about 11,000 to about 13,500 μg/m², from about 11,000 to about 13,000 μg/m², from about 11,000 to about 12,500 μg/m², from about 11,000 to about 12,000 μg/m², from about 11,000 to about 11,500 μg/m². In some embodiments, the prophylactically effective amount of the antibody comprises 10 to 14 days course of intravenous (IV) infusion of teplizumab at from about 11,000 to about 14,000 μg/m², from about 11,000 to about 13,500 μg/m², from about 11,000 to about 13,000 μg/m², from about 11,000 to about 12,500 μg/m², from about 11,000 to about 12,000 μg/m², from about 11,000 to about 11,500 μg/m². In some embodiments, the prophylactically effective amount of the antibody comprises 10 to 14 days course of intravenous (IV) infusion of teplizumab at from about 11,500 to about 14,000 μg/m², from about 11,500 to about 13,500 μg/m², from about 11,500 to about 13,000 μg/m², from about 11,500 to about 12,500 μg/m², from about 11,500 to about 12,000 μg/m². In some embodiments, the prophylactically effective amount of the antibody comprises 10 to 14 days course of intravenous (IV) infusion of teplizumab at from about 12,000 to about 14,000 μg/m², from about 12,000 to about 13,500 μg/m², from about 12,000 to about 13,000 μg/m², from about 12,000 to about 12,500 μg/m². In some embodiments, the prophylactically effective amount of the antibody comprises 10 to 14 days course of intravenous (IV) infusion of teplizumab at from about 12,500 to about 14,000 μg/m², from about 12,500 to about 13,500 μg/m², from about 12,500 to about 13,000 μg/m². In some embodiments, the prophylactically effective amount of the antibody comprises 10 to 14 days course of intravenous (IV) infusion of teplizumab at from about 13,000 to about 14,000 μg/m², from about 13,000 to about 13,500 μg/m². In some embodiments, the prophylactically effective amount of the antibody comprises 10 to 14 days course of intravenous (IV) infusion of teplizumab at from about 13,500 μg/m² to about 14,000 μg/m². In some embodiments, the prophylactically effective amount of the antibody comprises 10 to 14 days course of intravenous (IV) infusion of teplizumab at about 10,000 μg/m², 10,500 μg/m², 11,000 μg/m², 11.500 μg/m², 12,000 μg/m², 12,500 μg/m², 13,000 μg/m², 13,500 μg/m², or 14,000 μg/m². In some embodiments, the anti-CD3 antibody is or comprises teplizumab.

In some embodiments, the method comprises administering to the non-diabetic subject who is at risk for T1D a 14-day course IV infusion at about 60 μg/m², about 125 μg/m², about 250 μg/m², and about 500 μg/m², on days 1-4, respectively, and a dose of about 1,000 μg/m² on each of days 5-14. In some embodiments, the cumulative dose is about 10,935 μg/m².

In some embodiments, the method comprises administering to the non-diabetic subject who is at risk for T1D a 14-day course IV infusion at about 60 μg/m², about 125 μg/m², about 250 μg/m², and about 500 μg/m², on days 1-4, respectively, and a dose of about 1,030 μg/m² on each of days 5-14. In some embodiments, the cumulative dose is about 11,235 μg/m².

In some embodiments, the method comprises administering to the non-diabetic subject who is at risk for T1D a 14-day course IV infusion at about 100 μg/m², about 425 μg/m², about 850 μg/m², and about 850 μg/m², on days 1-4, respectively, and a dose of about 1,000 μg/m² on each of days 5-14. In some embodiments, the cumulative dose is about 12,225 μg/m².

In some embodiments, the method comprises administering to the non-diabetic subject who is at risk for T1D a 14-day course IV infusion at about 65 μg/m², about 125 μg/m², about 250 μg/m², and about 500 μg/m², on days 1-4, respectively, and a dose of about 1,070 μg/m² on each of days 5-14. In some embodiments, the cumulative dose is about 11,640 μg/m².

In some embodiments, the method comprises administering to a subject in need thereof a 14-day course IV infusion, wherein the daily dose is at about 65 μg/m², about 125 μg/m², about 250 μg/m², and about 500 μg/m², on days 1-4, respectively, and at about 1,030 μg/m² on each of days 5-14. In some embodiments, the cumulative dose is about 11,240 μg/m².

In some embodiments, the method comprises administering to a subject in need thereof a 14-day course IV infusion, wherein the daily dose is at about 60 μg/m², about 125 μg/m², about 250 μg/m², and about 500 μg/m², on days 1-4, respectively, and at about 1,000 μg/m² on each of days 5-14. In some embodiments, the cumulative dose is about 10,935 μg/m².

In some embodiments, the method comprises administering to a subject in need thereof a 14-day course IV infusion, wherein the daily dose is at about 60 μg/m², about 125 μg/m², about 250 μg/m², and about 500 μg/m², on days 1-4, respectively, and at about 1,030 μg/m² on each of days 5-14. In some embodiments, the cumulative dose is about 11,235 μg/m².

In some embodiments, the method comprises administering to subject in need thereof a 14-day course IV infusion, wherein the daily dose is at about 100 μg/m², about 425 μg/m², about 850 μg/m², and about 850 μg/m², on days 1-4, respectively, and at about 1,000 μg/m² on each of days 5-14. In some embodiments, the cumulative dose is about 12,225 μg/m².

In some embodiments, the method comprises administering to a subject in need thereof a 14-day course IV infusion, wherein the daily dose is at about 65 μg/m², about 125 μg/m², about 250 μg/m², and about 500 μg/m², on days 1-4, respectively, and a dose of about 1,070 μg/m² on each of days 5-14. In some embodiments, the cumulative dose is about 11,640 μg/m².

In some embodiments, the method comprises administering to a subject in need thereof a 14-day course IV infusion, wherein the daily dose is at about 65 μg/m², about 125 μg/m², about 250 μg/m², and about 500 μg/m², on days 1-4, respectively, and at about 1,030 μg/m² on each of days 5-14. In some embodiments, the cumulative dose is about 11,240 μg/m².

In some embodiment, the anti-CD3 antibody daily administration is missed, and dosing resumes by administering all remaining doses on consecutive days to complete the 14-day treatment course.

In some embodiments, the prophylactically effective amount delays median time to clinical diagnosis of T1D by at least 50%, at least 80%, or at least 90%, or at least 12 months, at least 18 months, at least 24 months, at least 36 months, at least 48 months, or at least 60 months, or longer.

In some embodiments, the course of dosing with the anti-CD3 antibody such as teplizumab can be repeated at 2 month, 4 month, 6 month, 8 month, 9 month, 10 month, 12 month, 15 month, 18 month, 24 month, 30 month, or 36 month intervals. In some embodiments, efficacy of the treatment with the anti-CD3 antibody such as teplizumab is determined as described herein, or as is known in the art, at 2 months, 4 months, 6 months, 9 months, 12 months, 15 months, 18 months, 24 months, 30 months, or 36 months subsequent to the previous treatment.

In some embodiments, a subject is administered one or more unit doses of approximately 0.5-50 μg/kg, approximately 0.5-40 μg/kg, approximately 0.5-30 μg/kg, approximately 0.5-20 μg/kg, approximately 0.5-15 μg/kg, approximately 0.5-10 μg/kg, approximately 0.5-5 μg/kg, approximately 1-5 μg/kg, approximately 1-10 μg/kg, approximately 20-40 μg/kg, approximately 20-30 μg/kg, approximately 22-28 μg/kg or approximately 25-26 μg/kg of the anti-CD3 antibody such as teplizumab to prevent, treat, ameliorate, mitigate, relieve, lighten, postpone, stop or halt one or more symptoms of T1D. In some embodiments, a subject is administered one or more unit doses of about 200 μg/kg, 178 μg/kg, 180 μg/kg, 128 μg/kg, 100 μg/kg, 95 μg/kg, 90 μg/kg, 85 μg/kg, 80 μg/kg, 75 μg/kg, 70 μg/kg, 65 μg/kg, 60 μg/kg, 55 μg/kg, 50 μg/kg, 45 μg/kg, 40 μg/kg, 35 μg/kg, 30 μg/kg, 26 μg/kg, 25 μg/kg, 20 μg/kg, 15 μg/kg, 13 μg/kg, 10 μg/kg, 6.5 μg/kg, 5 μg/kg, 3.2 μg/kg, 3 μg/kg, 2.5 μg/kg, 2 μg/kg, 1.6 μg/kg, 1.5 μg/kg, 1 μg/kg, 0.5 μg/kg, 0.25 μg/kg, 0.1 μg/kg, or 0.05 μg/kg of the anti-CD3 antibody such as teplizumab to prevent, treat, ameliorate, mitigate, relieve, lighten, postpone, stop or halt one or more symptoms of T1D.

In some embodiments, a subject is administered one or more doses of the anti-CD3 antibody such as teplizumab at about 5-1200 μg/m², for example, at about 60-1070 μg/m². In some embodiments, a subject is administered one or more unit doses of 1200 μg/m², 1150 μg/m², 1100 μg/m², 1050 μg/m², 1000 μg/m², 950 μg/m², 900 μg/m², 850 μg/m², 800 μg/m², 750 μg/m², 700 μg/m², 650 μg/m², 600 ug/m², 550 μg/m², 500 μg/m², 450 μg/m², 400 μg/m², 350 μg/m², 300 μg/m², 250 μg/m², 200 μg/m², 150 μg/m², 100 μg/m², 50 μg/m², 40 μg/m², 30 μg/m², 20 μg/m², 15 μg/m², 10 μg/m², or 5 μg/m² of the anti-CD3 antibody such as teplizumab to prevent, treat, slow the progression of, delay the onset of T1D, ameliorate, mitigate, relieve, lighten, postpone, stop or halt one or more symptoms of T1D.

In some embodiments, the subject is administered a treatment regimen comprising one or more doses of a prophylactically effective amount of the anti-CD3 antibody such as teplizumab, wherein the course of treatment is administered over 2 days, 3 days, 4 days, 5 days, 6 days, 7 days, 8 days, 9 days, 10 days, 11 days, 12 days, 13 days or 14 days. In some embodiments, the subject is administered a treatment regimen comprising one or more doses of a prophylactically effective amount of the anti-CD3 antibody such as teplizumab, wherein the course of treatment is administered over 14 days. In some embodiments, the treatment regimen comprises administering doses of the prophylactically effective amount every day, every 2nd day, every 3rd day or every 4th day. In some embodiments, the treatment regimen comprises administering doses of the prophylactically effective amount on Monday, Tuesday, Wednesday, Thursday of a given week and not administering doses of the prophylactically effective amount on Friday, Saturday, and Sunday of the same week until 14 doses, 13 doses, 12 doses, 11 doses, 10 doses, 9 doses, or 8 doses have been administered. In some embodiments, the dose administered is the same each day of the regimen.

In some embodiments, a subject is administered a treatment regimen comprising one or more doses of a prophylactically effective amount of the anti-CD3 antibody such as teplizumab, wherein the prophylactically effective amount is about 200 μg/kg/day, 175 μg/kg/day, 150 μg/kg/day, 125 μg/kg/day, 100 μg/kg/day, 95 μg/kg/day, 90 μg/kg/day, 85 μg/kg/day, 80 μg/kg/day, 75 μg/kg/day, 70 μg/kg/day, 65 μg/kg/day, 60 μg/kg/day, 55 μg/kg/day, 50 μg/kg/day, 45 μg/kg/day, 40 μg/kg/day, 35 μg/kg/day, 30 μg/kg/day, 26 μg/kg/day, 25 μg/kg/day, 20 μg/kg/day, 15 μg/kg/day, 13 μg/kg/day, 10 μg/kg/day, 6.5 μg/kg/day, 5 μg/kg/day, 3.2 μg/kg/day, 3 μg/kg/day, 2.5 μg/kg/day, 2 μg/kg/day, 1.6 μg/kg/day, 1.5 μg/kg/day, 1 μg/kg/day, 0.5 μg/kg/day, 0.25 μg/kg/day, 0.1 μg/kg/day, or 0.05 μg/kg/day; and/or wherein the prophylactically effective amount is about 1200 μg/m²/day, 1150 μg/m²/day, 1100 μg/m²/day, 1050 μg/m²/day, 1000 μg/m²/day, 950 μg/m²/day, 900 μg/m²/day, 850 μg/m²/day, 800 μg/m²/day, 750 μg/m²/day, 700 μg/m²/day, 650 μg/m²/day, 600 μg/m²/day, 550 μg/m²/day, 500 μg/m²/day, 450 μg/m²/day, 400 μg/m²/day, 350 μg/m²/day, 300 μg/m²/day, 250 μg/m²/day, 200 μg/m²/day, 150 μg/m²/day, 100 μg/m²/day, 50 μg/m²/day, 40 μg/m²/day, 30 μg/m²/day, 20 μg/m²/day, 15 μg/m²/day, 10 μg/m²/day, or 5 μg/m²/day.

In some embodiments, the intravenous dose of about 1200 μg/m² or less, 1150 μg/m² or less, 1100 μg/m² or less, 1050 μg/m² or less, 1000 μg/m² or less, 950 μg/m² or less, 900 μg/m² or less, 850 μg/m² or less, 800 μg/m² or less, 750 μg/m² or less, 700 μg/m² or less, 650 μg/m² or less, 600 μg/m² or less, 550 μg/m² or less, 500 μg/m² or less, 450 μg/m² or less, 400 μg/m² or less, 350 μg/m² or less, 300 μg/m² or less, 250 μg/m² or less, 200 μg/m² or less, 150 μg/m² or less, 100 μg/m² or less, 50 μg/m² or less, 40 μg/m² or less, 30 μg/m² or less, 20 μg/m² or less, 15 μg/m² or less, 10 μg/m² or less, or 5 μg/m² or less of the anti-CD3 antibody such as teplizumab is administered over about 24 hours, about 22 hours, about 20 hours, about 18 hours, about 16 hours, about 14 hours, about 12 hours, about 10 hours, about 8 hours, about 6 hours, about 4 hours, about 2 hours, about 1.5 hours, about 1 hour, about 50 minutes, about 40 minutes, about 30 minutes, about 20 minutes, about 10 minutes, about 5 minutes, about 2 minutes, about 1 minute, about 30 seconds or about 10 seconds to prevent, treat, ameliorate, mitigate, relieve, lighten, postpone, stop or halt one or more symptoms of type 1 diabetes. The total dosage over the duration of the regimen can be greater than about 9,500, 10,000, 10,500, 11,000, 11,500, 12,000, 12,500, 13,000 or 13,500 ug/m². The total dosage over the duration of the regimen, in some embodiments, is from about 9,500 to about 14,000 μg/m², from about 9,500 to about 13,500 μg/m², from about 9,500 to about 13,000 μg/m², from about 9,500 to about 12,500 μg/m², from about 9,500 to about 12,000 μg/m², from about 9,500 to about 11,500 μg/m², from about 9,500 to about 11,000 μg/m², from about 9,500 to about 10,500 μg/m², from about 9,500 to about 10,000 μg/m². The total dosage over the duration of the regimen, in some embodiments, is from about 10,500 to about 14,000 μg/m², from about 10,500 to about 13,500 μg/m², from about 10,500 to about 13,000 μg/m², from about 10,500 to about 12,500 μg/m², from about 10,500 to about 12,000 μg/m², from about 10,500 to about 11,500 μg/m², from about 10,500 to about 11,000 μg/m². For example, the total dosage over the duration of the regimen is about 10,935 μg/m², about 11,235 μg/m², about 11,240 μg/m², about 11,640 μg/m², or about 12,225 μg/m².

In some embodiments, the dose escalates over the first fourth, first half or first ⅔ of the doses (e.g., over the first 2, 3, 4, 5, or 6 days of a 10, 12, 14, 16, 18 or 20-day regimen of one dose per day) of the treatment regimen until the daily prophylactically effective amount of the anti-CD3 antibody such as teplizumab is achieved. For example, the dose can escalate over 3 or 4 days of a 14-day regimen. In some embodiments, a subject is administered a treatment regimen comprising one or more doses of a prophylactically effective amount of the anti-CD3 antibody such as teplizumab, wherein the prophylactically effective amount is increased by, e.g., about 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg each day; or increased by, e.g., 1 μg/m², 5 μg/m², 10 μg/m², 15 μg/m², 20 μg/m², 30 μg/m², 40 μg/m², 50 μg/m², 60 μg/m², 70 μg/m², 80 μg/m², 90 μg/m², 100 μg/m², 150 μg/m², 200 μg/m², 250 μg/m², 300 μg/m², 350 μg/m², 400 μg/m², 450 μg/m², 500 μg/m², 550 μg/m², 600 μg/m², or 650 μg/m², each day as treatment progresses. In some embodiments, a subject is administered a treatment regimen comprising one or more doses of a prophylactically effective amount of the anti-CD3 antibody such as teplizumab, wherein the prophylactically effective amount is increased by a factor of 1.25, a factor of 1.5, a factor of 2, a factor of 2.25, a factor of 2.5, or a factor of 5 until the daily prophylactically effective amount of the anti-CD3 antibody such as teplizumab is achieved.

In some embodiments, a subject is intramuscularly administered one or more doses of a about 200 μg/kg or less, preferably about 175 μg/kg or less, 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.2 μg/kg or less of the anti-CD3 antibody such as teplizumab, otelixizumab or foralumab, to prevent, treat, ameliorate, mitigate, relieve, lighten, postpone, stop or halt one or more symptoms of T1D.

In some embodiments, a subject is subcutaneously administered one or more doses of a about 200 ug/kg or less, preferably about 175 μg/kg or less, 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.2 μg/kg or less of the anti-CD3 antibody such as teplizumab, otelixizumab or foralumab, to prevent, treat, ameliorate, mitigate, relieve, lighten, postpone, stop or halt one or more symptoms of T1D.

In some embodiments, a subject is intravenously administered one or more doses of a about 100 ug/kg or less, preferably about 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, 1 ug/kg or less, 0.5 ug/kg or less, or 0.2 ug/kg or less of the anti-CD3 antibody such as teplizumab, otelixizumab or foralumab, to prevent, treat, ameliorate, mitigate, relieve, lighten, postpone, stop or halt one or more symptoms of T1D. In some embodiments, the intravenous dose of about 100 μg/kg or less, 95 μg/kg or less, 90 μg/kg or less, 85 μg/kg or less, 80 μg/kg or less, 75 μg/kg or less, 70 μg/kg or less, 65 u μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.2 μg/kg or less of the anti-CD3 antibody such as teplizumab, otelixizumab or foralumab, is administered over about 6 hours, about 4 hours, about 2 hours, about 1.5 hours, about 1 hour, about 50 minutes, about 40 minutes, about 30 minutes, about 20 minutes, about 10 minutes, about 5 minutes, about 2 minutes, about 1 minute, about 30 seconds or about 10 seconds to prevent, treat, ameliorate, mitigate, relieve, lighten, postpone, stop or halt one or more symptoms of T1D.

In some embodiments, a subject is orally administered one or more doses of a about 100 μg/kg or less, preferably about 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.2 μg/kg or less of the anti-CD3 antibody such as teplizumab, otelixizumab or foralumab, to prevent, treat, ameliorate, mitigate, relieve, lighten, postpone, stop or halt one or more symptoms of T1D. In some embodiments, the oral dose of about 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.2 μg/kg or less of the anti-CD3 antibody such as teplizumab, otelixizumab or foralumab, is administered over about 6 hours, about 4 hours, about 2 hours, about 1.5 hours, about 1 hour, about 50 minutes, about 40 minutes, about 30 minutes, about 20 minutes, about 10 minutes, about 5 minutes, about 2 minutes, about 1 minute, about 30 seconds or about 10 seconds to prevent, treat, ameliorate, mitigate, relieve, lighten, postpone, stop or halt one or more symptoms of T1D.

In some embodiments in which escalating doses are administered for the first days of the dosing regimen, the dose on day 1 of the regimen is about 5-150 μg/m²/day, preferably about 55-150 μg/m²/day, for example about 60-100 μg/m²/day and escalates to the daily dose as recited immediately above by day 3, 4, 5, 6 or 7. In some embodiments, on day 1, the subject is administered a dose of approximately 60 μg/m²/day, on day 2 approximately 125 μg/m²/day, on day 3 approximately 250 μg/m²/day, on day 4 approximately 500 μg/m²/day and on subsequent days of the regimen (e.g., days 5-14) 1,000 μg/m²/day. In some embodiments, on day 1, the subject is administered a dose of approximately 60 μg/m²/day, on day 2 approximately 125 μg/m²/day, on day 3 approximately 250 μg/m²/day, on day 4 approximately 500 μg/m²/day and on subsequent days of the regimen (e.g., days 5-14) 1,030 μg/m²/day. In some embodiments, on day 1, the subject is administered a dose of approximately 100 μg/m²/day, on day 2 approximately 425 μg/m²/day, on day 3 approximately 850 μg/m²/day, on day 4 approximately 850 μg/m²/day and on subsequent days of the regimen (e.g., days 5-14) 1,000 μg/m²/day. In some embodiments, on day 1, the subject is administered a dose of approximately 60 μg/m²/day, on day 2 approximately 125 μg/m²/day, on day 3 approximately 250 μg/m²/day, on day 4 approximately 500 μg/m²/day and on subsequent days of the regimen (e.g., days 5-14) 1,070 μg/m²/day.

In some embodiments, the initial dose is ¼, to ½, to equal to the daily dose at the end of the regimen but is administered in portions at intervals of 6, 8, 10 or 12 hours. For example, a 13 μg/kg/day dose is administered in four doses of 3-4 μg/kg at intervals of 6 hours to reduce the level of cytokine release caused by administration of the antibody. In some embodiments, to reduce the possibility of cytokine release and other adverse effects, the first 1, 2, 3, or 4 doses or all the doses in the regimen are administered more slowly by intravenous administration. For example, a dose of 51 μg/m²/day may be administered over about 5 minutes, about 15 minutes, about 30 minutes, about 45 minutes, about 1 hour, about 2 hours, about 4 hours, about 6 hours, about 8 hours, about 10 hours, about 12 hours, about 14 hours, about 16 hours, about 18 hours, about 20 hours, and about 22 hours. In some embodiments, the dose is administered by slow infusion over a period of, e.g., 20 to 24 hours. In some embodiments, the dose is infused in a pump, preferably increasing the concentration of antibody administered as the infusion progresses.

In some embodiments, a set fraction of the doses for, for example, the 60 μg m²/day to 1000 μg/m²/day or the 65 μg m²/day to 1030 μg/m²/day regimen described above is administered in escalating doses. In some embodiments, the fraction is 1/10, ¼, ⅓, ½, ⅔ or ¾ of the daily doses of the regimens described above.

In some embodiments, the anti-CD3 antibody such as teplizumab, otelixizumab or foralumab, is not administered by daily doses over a number of days, but is rather administered by infusion in an uninterrupted manner over a minimum of 30 minutes, for example 30 minutes, 1 hours, 2 hours, 3 hours, 4 hours, 5 hours, 6 hours, 7 hours, 8 hours, 9 hours, 10 hours, 12 hours, 15 hours, 18 hours, 20 hours, 24 hours, 30 hours or 36 hours. The infusion may be constant or may start out at a lower dosage for, for example, the first 30 minutes, 1 hour, 2 hours, 3 hours, 4 hours, 5 hours, 6 hours, 7 hours or 8 hours of the infusion and then increase to a higher dosage thereafter. Over the course of the infusion, the patient receives a dose equal to the amount administered in the 5 to 20-day regimens set forth above (for example the 14-day regimen set forth above). For example, a cumulative dose of approximately or greater than 9500 μg/m², 10000 μg/m², 10500 μg/m², 11000 μg/m², 11500 μg/m², 12000 μg/m², 12500 μg/m², 13000 μg/m², 13500 μg/m² or 14000 μg/m² is administered by infusion to the subject in need thereof. For example, a cumulative dose of teplizumab of approximately or greater than 11,240 μg/m² is administered by infusion to the subject in need thereof over a period of at least 30 minutes. In particular, the speed and duration of the infusion is designed to minimize the level of free anti-CD3 antibody such as teplizumab, otelixizumab or foralumab in the subject after administration. In some embodiments, the level of free anti-CD3 antibody such as teplizumab should not exceed 200 ng/ml free antibody. In addition, the infusion is designed to achieve a combined T cell receptor coating and modulation of at least 50%, 60%, 70%, 80%, 90%, 95% or of 100%.

In some embodiments, the subject in need thereof is administered prior to the administering of the course IV infusion (for example the 14-days course IV infusion) on each of days 1-5 an effective amount of pain reliever (such as a nonsteroidal anti-inflammatory drug (NSAID), acetaminophen), an antihistamine, an antiemetic or a combination thereof. In some embodiments, the pain reliever (such as a nonsteroidal anti-inflammatory drug (NSAID), acetaminophen), an antihistamine, an antiemetic or a combination thereof can be administered on each of days 1-5, 1-6, 1-7, 1-8, 1-9, 1-10, 1-11, 1-12, 1-13, 1-14 or during the duration of the treatment with the anti-CD3 antibodies. In some embodiments, the NSAID, acetaminophen, antihistamine, antiemetic or combination thereof is administered orally. In some embodiments, the NSAID, acetaminophen, antihistamine, antiemetic or combination thereof is administered intravenously. In some embodiments, antipyretics, antihistamines and/or antiemetics are administered to the subject in need thereof to mitigate cytokine release syndrome. In some embodiments, the liver enzymes are monitored and treatment with the anti-CD3 antibodies is discontinued or paused in subjects developing elevated ALT or AST more than 5 times the upper limit of normal.

In some embodiments, the anti-CD3 antibody such as teplizumab, otelixizumab or foralumab is administered chronically to treat, prevent, or slow or delay the onset or progression, or ameliorate, mitigate, relieve, lighten, postpone, stop or halt one or more symptoms of type 1 diabetes. For example, in some embodiments, a low dose of the anti-CD3 antibody such as teplizumab is administered once a month, twice a month, three times per month, once a week or even more frequently either as an alternative to the 6 to 14-day dosage regimen discussed above or after administration of such a regimen to enhance or maintain its effect. Such a low dose may be anywhere from about 1 μg/m² to about 100 μg/m², such as approximately 5 μg/m², 10 μg/m², 15 μg/m², 20 μg/m², 25 μg/m², 30 μg/m², 35 μg/m², 40 μg/m², 45 μg/m², 50 μg/m², 55 μg/m², 60 μg/m², 65 μg/m², 70 μg/m², 75 μg/m², 80 μg/m², 85 μg/m², 90 μg/m², 95 μg/m² or 100 μg/m².

In some embodiments, the anti-CD3 antibody such as teplizumab, otelixizumab or foralumab is administered by infusion in medical facilities, outpatient infusion centers. Yet in other embodiments, the anti-CD3 antibody such as teplizumab, otelixizumab or foralumab is administered by infusion in a home setting. Home infusion therapy involves the administration of the therapeutic agents, for example, anti-CD3 antibody using intravenous, or subcutaneous routes, in the patient's home rather than in a physician's office or hospital. Infusion therapies in the home can be administered by a home health care worker or by a patient himself. In some embodiments, a health care worker having some training in the operation of infusion equipment and the administration of anti-CD3 antibody can provide the patient with self-administration training and all the necessary equipment and/or supplies needed for the administration.

In some embodiments, the subject may be re-dosed at some time subsequent to administration of the anti-CD3 antibody such as teplizumab, otelixizumab or foralumab dosing regimen, for example, based upon one or more physiological parameters or may be done as a matter of course. Such redosing may be administered and/or the need for such redosing evaluated 2 months, 4 months, 6 months, 8 months, 9 months, 1 year, 15 months, 18 months, 2 years, 30 months or 3 years after administration of a dosing regimen and may include administering a course of treatment every 6 months, 9 months, 1 year, 15 months, 18 months, 2 years, 30 months or 3 years indefinitely.

Some embodiments relate to an anti-CD3 antibody for use in a method of preventing or delaying the onset of clinical type 1 diabetes (T1D), comprising administering a prophylactically effective amount of an anti-CD3 antibody to a non-diabetic subject who is at risk of T1D, wherein the prophylactically effective amount has a cumulative dose of about 10,500 μg/m² to about 14,000 μg/m²; and determining, prior to or after the administering step, that the non-diabetic subject has more than about 10% TIGIT+KLRG1+CD8+ T-cells in all CD3+ T cells, which is indicative of successful prevention or delay of the onset of clinical T1D.

Aspects of the disclosure relate to a method of preventing or delaying onset of clinical type 1 diabetes (T1D), comprising administering a prophylactically effective amount of an anti-CD3 antibody to a non-diabetic subject who is at risk of T1D, wherein the prophylactically effective amount has a cumulative dose of from about 10,500 μg/m² to about 14,000 μg/m².

In some embodiments, the subject is 8 years of age or older. In some embodiments, the subject is 5 years of age or older. In some embodiments, the subject is 3 years of age or older. In some embodiments, the subject is 1 year of age or older. In some embodiments, the subject is 1 year old or younger.

In some embodiments, the non-diabetic subject is a relative of a patient with T1D.

In some embodiments, the method further comprises determining that the non-diabetic subject (1) is negative for zinc transporter 8 (ZnT8) antibodies, (2) is HLA-DR4+, and/or (3) is not HLA-DR3+. In some embodiments, the non-diabetic subject does not have antibodies against ZnT8. In some embodiments, the non-diabetic subject is HLA-DR4+ and is not HLA-DR3+.

In some embodiments, the non-diabetic subject has 2 or more diabetes-related autoantibodies selected from islet cell antibodies (ICA), insulin autoantibodies (IAA), and antibodies to glutamic acid decarboxylase (GAD), tyrosine phosphatase (IA-2/ICA512) or ZnT8.

In some embodiments, the non-diabetic subject has abnormal glucose tolerance on oral glucose tolerance test (OGTT). In some embodiments, the abnormal glucose tolerance on OGTT is a fasting glucose level of 110-125 mg/dL, or 2 hour plasma of ≥140 and <200 mg/dL, or an intervening glucose value at 30, 60, or 90 minutes on OGTT>200 mg/dL.

In some embodiments, the anti-CD3 antibody is selected from teplizumab, otelixizumab or foralumab. In some embodiments, the anti-CD3 antibody is teplizumab.

In some embodiments, the prophylactically effective amount comprises a 14-day course of subcutaneous (SC) injection or intravenous (IV) infusion or oral administration of the anti-CD3 antibody with a cumulative dose of from about 11,000 μg/m² to about 14,000 μg/m².

In some embodiments, the method comprises administering a 14-day course IV infusion at about 60 μg/m² on day 1, about 125 μg/m² on day 2, about 250 μg/m² on day 3, and about 500 μg/m² on day 4, and a dose of about 1,000 μg/m² on each of days 5-14.

In some embodiments, the method comprises administering a 14-day course IV infusion at about 60 μg/m², about 125 μg/m² on day 1, about 250 μg/m² on day 2, and about 500 μg/m² on day 3, and a dose of about 1,030 μg/m² on each of days 5-14.

In some embodiments, the method comprises administering a 14-day course IV infusion at about 100 μg/m² on day 1, about 425 μg/m² on day 2, about 850 μg/m² on day 3, about 850 μg/m² on day 4, and a dose of about 1,000 μg/m² on each of days 5-14.

In some embodiments, the method comprises administering a 14-day course IV infusion at about 65 μg/m² on day 1, about 125 μg/m² on day 2, about 250 μg/m² on day 3, and about 500 μg/m² on day 4, and a dose of about 1,070 μg/m² on each of days 5-14.

In some embodiments, the method comprises administering a 14-day course IV infusion at about 65 μg/m² on day 1, about 125 μg/m² on day 2, about 250 μg/m² on day 3, and about 500 μg/m² on day 4, and a dose of about 1,030 μg/m² on each of days 5-14.

In some embodiments, the prophylactically effective amount delays median time to clinical diagnosis of T1D by at least 50%, at least 80%, or at least 90%. In some embodiments, the prophylactically effective amount delays median time to clinical diagnosis of T1D by at least 12 months, at least 18 months, at least 24 months, at least 36 months, at least 48 months, or at least 60 months.

In some embodiments, the method further comprises determining, prior to or after the administering step, that the non-diabetic subject has more than about 10% TIGIT+KLRG1+CD8+ T-cells in all CD3+ T cells, which is indicative of successful prevention or delay of the onset of clinical T1D. In some embodiments, the determining of TIGIT+KLRG1+CD8+ T-cells is by flow cytometry. In some embodiments, the method further comprises determining a decrease in a percentage of CD8+ T cells expressing proliferation markers Ki67 and/or CD57.

Aspects of the disclosure relate to a method of preventing or delaying onset of clinical type 1 diabetes (T1D), comprising administering to a non-diabetic subject 8 years of age or older who is at risk of T1D a 14-day course IV infusion of teplizumab at about 65 μg/m² on day 1, about 125 μg/m² on day 2, about 250 μg/m² on day 3, and about 500 μg/m² on day 4, and a dose of about 1,030 μg/m² on each of days 5-14.

Aspects of the disclosure relate to a method of delaying onset of Stage 3 type 1 diabetes (T1D), comprising administering to a subject in need thereof who has a diagnosis of stage 2 T1D a 14-day course IV infusion of teplizumab at about 65 μg/m² on day 1, about 125 μg/m² on day 2, about 250 μg/m² on day 3, and about 500 μg/m² on day 4, and a dose of about 1,030 μg/m² on each of days 5-14.

In some embodiments, the subject in need thereof is an adult. In some embodiments, the subject in need thereof is a pediatric subject 8 years of age or older. In some embodiments, the subject in need thereof is a pediatric subject 5 years of age or older. In some embodiments, the subject in need thereof is a pediatric subject 3 years of age or older. In some embodiments, the subject in need thereof is a pediatric subject 1 year of age or older. In some embodiments, the subject in need thereof is a pediatric subject 1 year old or younger.

In some embodiments, the method comprises documenting at least two positive pancreatic islet autoantibodies in the subject who has dysglycemia without overt hyperglycemia before administering the 14-day course. In some embodiments, the subject in need thereof has dysglycemia without overt hyperglycemia and has two or more pancreatic islet autoantibodies. In some embodiments, the two or more pancreatic islet autoantibodies comprise islet cell antibodies (ICA), insulin autoantibodies (IAA), and antibodies to glutamic acid decarboxylase (GAD), tyrosine phosphatase (IA-2/ICA512) or ZnT8. In some embodiments, the method comprises administering on at least each of days 1-5, and prior to the administering of the 14-day course IV infusion, an effective amount of a nonsteroidal anti-inflammatory drug (NSAID), acetaminophen, an antihistamine, an antiemetic or a combination thereof. In some embodiments, the method comprises administering orally the NSAID, acetaminophen, antihistamine, antiemetic or combination thereof. In some embodiments, the method comprises administering the 14-day course IV infusion once daily for 14 consecutive days over a period of at least 30 minutes.

Aspects of the disclosure relate to a method of prognosing responsiveness of an anti-CD3 antibody in preventing or delaying onset of type 1 diabetes (T1D), the method comprising administering a prophylactically effective amount of an anti-CD3 antibody to a non-diabetic subject who is at risk of T1D, wherein the prophylactically effective amount has a cumulative dose of about 10,500 μg/m² to about 14,000 μg/m²; and determining C-peptide area under the curve (AUC): glucose AUC ratio, wherein an increase in the ratio indicates responsiveness to the anti-CD3 antibody and/or non-progression to clinical T1D.

Aspects of the disclosure relate to an anti-CD3 antibody for use in a method of preventing or delaying onset of clinical type 1 diabetes (T1D), comprising: administering a prophylactically effective amount of an anti-CD3 antibody to a non-diabetic subject who is at risk of T1D, wherein the prophylactically effective amount has a cumulative dose of about 10,500 μg/m² to about 14,000 μg/m².

Aspects of the disclosure relate to a teplizumab composition for use in a method of delaying onset of Stage 3 type 1 diabetes (T1D), comprising administering to a subject in need thereof who has a diagnosis of stage 2 T1D a 14-day course IV infusion at about 65 μg/m² on day 1, about 125 μg/m² on day 2, about 250 μg/m² on day 3, and about 500 μg/m² on day 4, and a dose of about 1,030 μg/m² on each of days 5-14.

### EXAMPLES

### Example 1

Type 1 diabetes (T1D) is an autoimmune disease characterized by T-cell mediated destruction of insulin producing beta cells within the pancreatic islets of Langerhans. Longitudinal observational studies over more than 30 years have described the progression of the autoimmune disease from the first appearance of autoantibodies until beta cell function is critically impaired and the clinical diagnosis, often with ketoacidosis, occurs (1-5). T1D is associated with a need for lifelong exogenous insulin administration for survival, increased morbidity and mortality due to immediate (e.g. hypoglycemia) and long-term complications (e.g. vascular, renal, and eye disease), and reduced life-span, life impairments, and considerable health-care-related costs (6-9). Thus, approaches to prevent progression to clinical T1D before irremediable beta cell destruction and insulin deficiency, are of paramount importance.

Changes in beta cell function precede the clinical diagnosis of T1D and have been studied in natural history cohorts of individuals who are identified as at-risk for the disease based on the presence of islet autoantibodies (10-12). Some studies suggest an ongoing and intermittently progressive decline in beta cell function, that begins years before clinical diagnosis at a time when glucose tolerance is normal. During this period there are signs of ongoing autoimmunity: Based on the findings of the natural history, individuals with two or more islet autoantibodies have been classified as stages of T1D, with further specification according to the level of metabolic dysfunction: Stage 1 prior to glucose abnormalities, Stage 2 with dysglycemia during an oral glucose tolerance test (OGTT), and Stage 3 at clinical presentation with hyperglycemia (2, 13, 14). However, the relationships between changes in beta cell function and clinical disease remain poorly defined. It is known, for example, that glucose tolerance, defined through responses to an oral glucose tolerance test (OGTT), may fluctuate between abnormal and normal values within an individual who is at risk (15, 16). In addition, OGTT glucose tolerance classifications used to designate a clinical diagnosis, and beta cell function, measured by C-peptide responses to a metabolic challenge, may not be closely related and at diagnosis using an OGTT, many individuals have clinically meaningful C-peptide responses (15-18).

The following are dosing regimens matching AUC_{inf}, Cₘₐₓ and C_{trough13} (trough concentration prior to Day 14 dosing) exposure parameters of the reference regimen (Herold 14-day regimen used in Protégé and TN-10 studies, Herold et al NEJM 2019).

Individual (conditional) PK simulations were performed using Provention Bio Model 363 (Regimen A, B and C), Model 372 (Regimen D) and individual model parameters from the corresponding arms of the PROTECT Phase 3 study of teplizumab in newly-diagnosed T1D patients.

Typical (average) simulations for comparison of exposure values were also performed.

More than 30 regimens based on the Herold 14-day regimen or PROTECT regimen were simulated and were compared to the reference regimen. Alternative dosage regimens are presented in Table 1.

TABLE 1
Regimens Used in Clinical Studies and Proposed Alternative Regimens for anti-CD3
antibody

| | | | | | |
|---|---|---|---|---|---|
| | | | | | Cumulative Dose |
| Day 1 | Day 2 | Day 3 | Day 4 | Days 5-14 | (μg/m²) |

REGIMENS USED IN CLINICAL STUDIES

| | | | | | | |
|---|---|---|---|---|---|---|
| TN-10 and Protégé | 51 | 103 | 207 | 413 | 826 | 9,034 |
| Herold 14-day | | | | | | |
| regimen | | | | | | |
| PROTECT 12-day | 106 | 425 | 850 | 850 | 850 | 9,031 |
| regimen | | | | | (D5-12) | |

PROPOSED ALTERNATIVE REGIMENS

| | | | | | | |
|---|---|---|---|---|---|---|
| Herold-based | | | | | | |
| Regimen A | 60 | 125 | 250 | 500 | 1000 | 10,935 |
| Regimen B | 60 | 125 | 250 | 500 | 1030 | 11,235 |
| Regimen D | 65 | 125 | 250 | 500 | 1030 | 11,240 |
| PROTECT-based | | | | | | |
| Regimen C | 100 | 425 | 850 | 850 | 1000 | 12,225 |

Dosing in μg/m² via 30-minute infusion

Each of these regimens includes a 4-day ramp-up followed by 10 consecutive days of maintenance dosing, with an infusion duration of 30 minutes. As in previous T1D regimens, the ramp-up is important to avoid the occurrence of acute events such as those related to cytokine release.
Regimen A: Incorporates the backbone of the TN-10 (Herold 14-day) 4-day ramp-up and maintenance doses, with an approximate 18-20% upward adjustment for each daily dose. This regimen meets the bioequivalence criteria for the three PK parameters under both conditional and typical (average) simulations with the exception that the average (typical) simulation ratio for C_{trough13} is slightly less than 0.80 (0.779). (Table 2). The cumulative dose for this regimen is 10,935 μg/m² (approximately 21% higher compared to the Herold 14-day regimen).
   Day 1: 60 μg/m²
   Day 2: 125 μg/m²
   Day 3: 250 μg/m²
   Day 4: 500 μg/m²
   Days 5-14: 1000 μg/m²
Regimen B: Incorporates the backbone of the TN-10 (Herold 14-day) regimen and an approximate 18-25% upward adjustment for each daily dose; this has a similar 4-day ramp-up as in Regimen A, but the maintenance dose is 1030 μg/m². This regimen meets all bioequivalence criteria for AUC_{inf}, Cₘₐₓ and C_{trough13} under both the conditional and typical (average) simulations (Table 2). The cumulative dose for this regimen is 11,235 μg/m² (approximately 24% higher compared to the Herold 14-day regimen).
   Day 1: 60 μg/m²
   Day 2: 125 μg/m²
   Day 3: 250 μg/m²
   Day 4: 500 μg/m²
   Days 5-14: 1030 μg/m²
Regimen D: Incorporates the backbone of the TN-10 (Herold 14-day) regimen and an approximate 18-25% upward adjustment for each daily dose; this has a similar 4-day ramp-up as in Regimen A, but the Day 1 dose is 65 μg/m² and the maintenance dose is 1030 μg/m². This regimen meets all bioequivalence criteria for AUC_{inf}, Cₘₐₓ and C_{trough13} under both the conditional and typical (average) simulations (Table 2). The cumulative dose for this regimen is 11,240 μg/m² (approximately 24% higher compared to the Herold 14-day regimen).
   Day 1: 65 μg/m²
   Day 2: 125 μg/m²
   Day 3: 250 μg/m²
   Day 4: 500 μg/m²
   Days 5-14: 1030 μg/m²
Regimen C: Incorporates the backbone of the PROTECT 12-day regimen with a 2-day ramp-up, with minor adjustment for the Day 1 dose from 106 μg/m² to 100 μg/m² to simplify dose calculations and preparations; the two doses of 850 μg/m² on Day 3 and Day 4 replicate the PROTECT regimen before the administration of the maintenance dose of 1000 μg/m². This regimen meets 80-125% bioequivalence criteria for AUC_{inf}, Cₘₐₓ and C_{trough13} under both the conditional and typical (average) simulations (Table 2). The cumulative dose for this regimen is 12,225 μg/m² (approximately 35% higher compared to the Herold 14-day regimen).
   Day 1: 100 μg/m²
   Day 2: 425 μg/m²
   Day 3: 850 μg/m²
   Day 4: 850 μg/m²
   Days 5-14: 1000 μg/m²

TABLE 2
Comparison of Alternative Regimens to Reference Regimen: Conditional and
Typical (average) Simulations

| | AUC_{inf} | | | Cₘₐₓ | | | C_{trough13} | | |
|---|---|---|---|---|---|---|---|---|---|
| | (ng*day/mL) | | | (ng/ml) | | | (ng/ml) | | |
| | Simulation Method | | | | | | | | |
| | Conditional | | | Conditional | | | Conditional | | |
| | Ratio | | Typical | Ratio | | Typical | Ratio | | Typical |
| | Geo | | (average) | Geo | | (average) | Geo | | (average) |
| Regimens | Mean | 90% CI | Ratio | Mean | 90% CI | Ratio | Mean | 90% CI | Ratio |
| Regimen A | 0.958 | 0.885- | 0.859 | 1.07 | 1.03- | 1.008 | 0.883 | 0.803- | 0.779 |
| | | 1.04 | | | 1.11 | | | 0.971 | |
| Regimen B | 0.99 | 0.911- | 0.885 | 1.11 | 1.07- | 1.042 | 0.917 | 0.834- | 0.809 |
| | | 1.07 | | | 1.15 | | | 1.01 | |
| Regimen C | 1.08 | 1.0-1.17 | 0.97 | 1.09 | 1.05- | 1.028 | 0.934 | 0.849- | 0.823 |
| | | | | | 1.14 | | | 1.03 | |
| Regimen D | 0.991 | 0.915- | 0.88 | 1.11 | 1.07- | 1.037 | 0.917 | 0.833- | 0.801 |
| | | 1.07 | | | 1.15 | | | 1.01 | |

Typical simulations performed using median BSA and weight of subjects in the PROTECT study and no anti-drug antibodies (ADA)

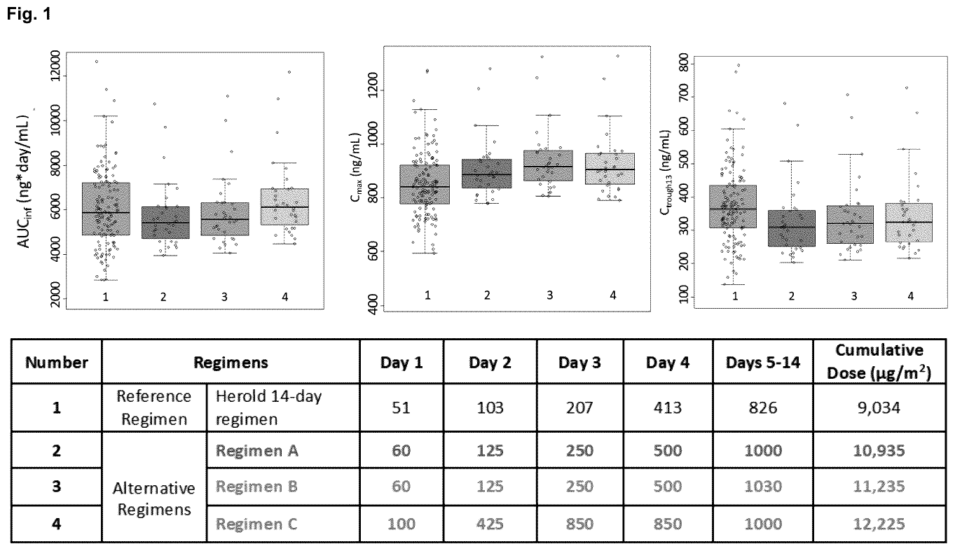

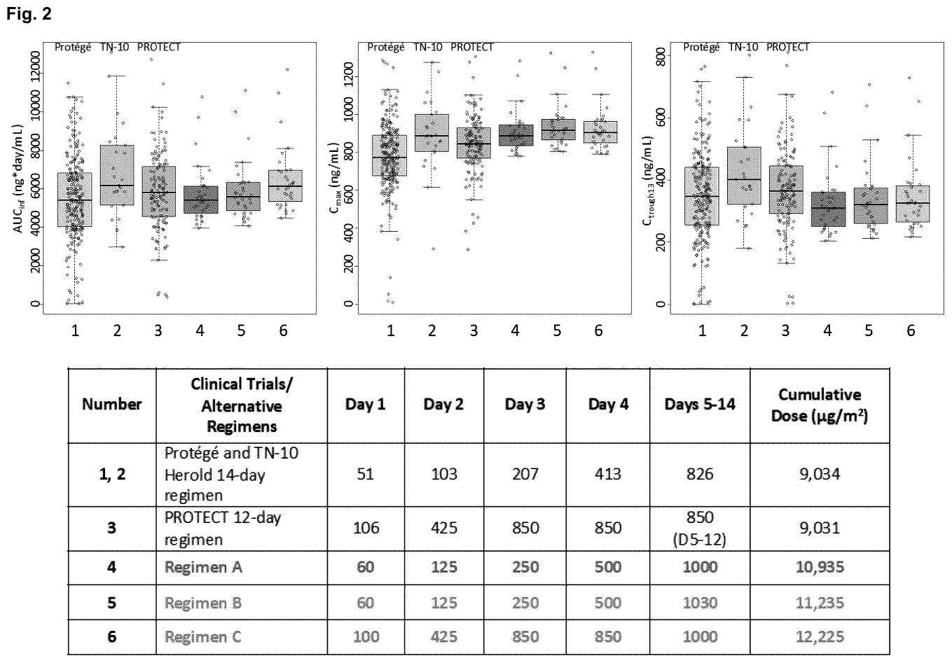

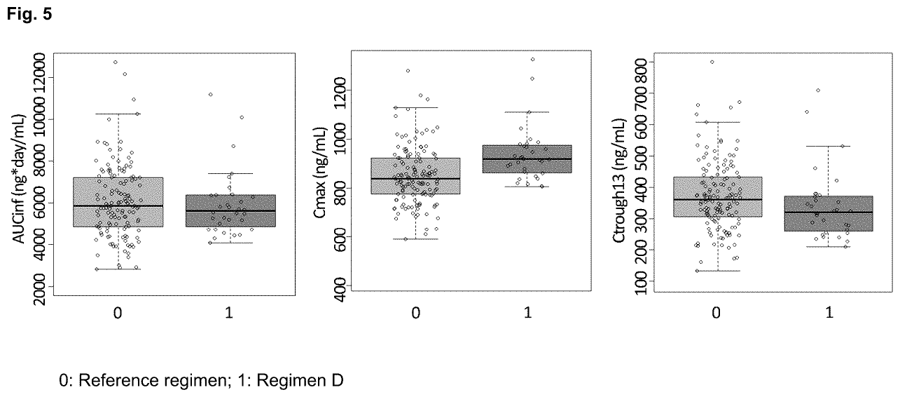

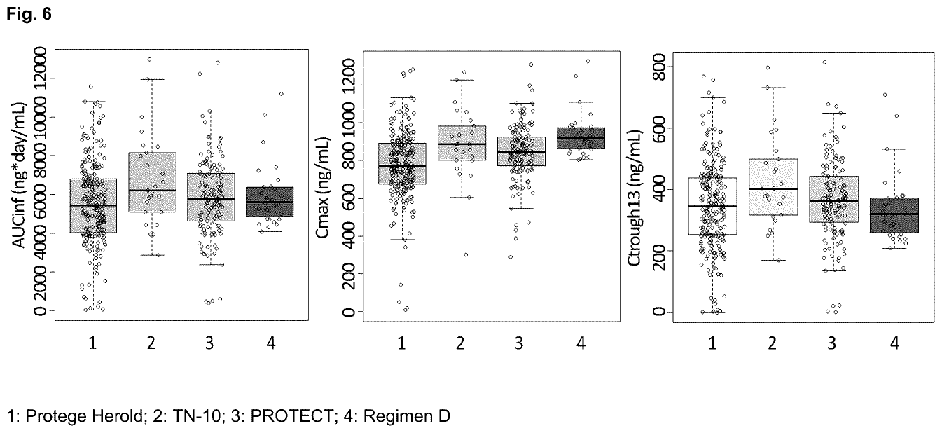

A comparison of the distribution of exposures (AUC_{inf}, Cₘₐₓ and C_{trough13}) for the reference regimen (Herold 14-day) and the anti-CD3 antibody exposures predicted for the alternative regimens are shown in FIG. 1 and FIG. 5. A comparison of the distribution of exposures (AUC_{inf}, Cₘₐₓ and C_{trough13}) for the Protégé and TN-10 regimen (Herold 14-day) and the anti-CD3 antibody exposures predicted for the alternative regimens are shown in FIG. 2 and FIG. 6.

### Safety Considerations

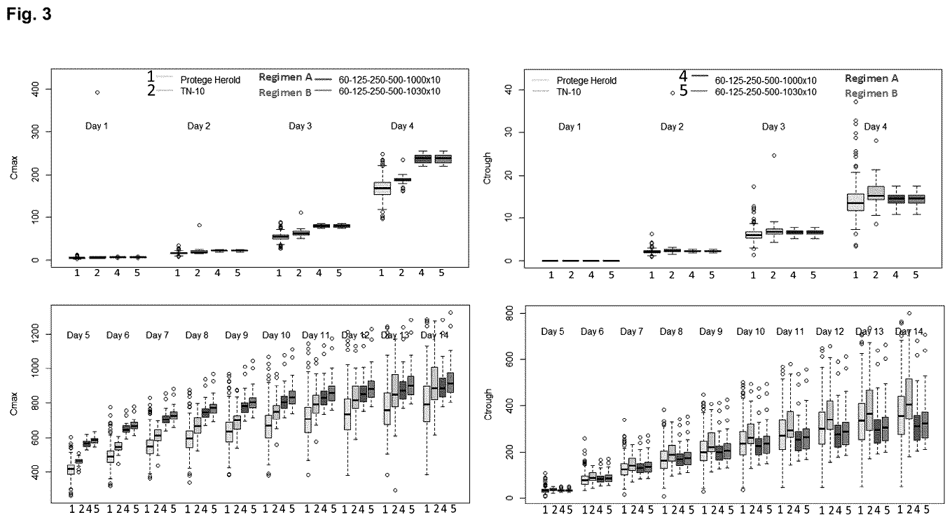

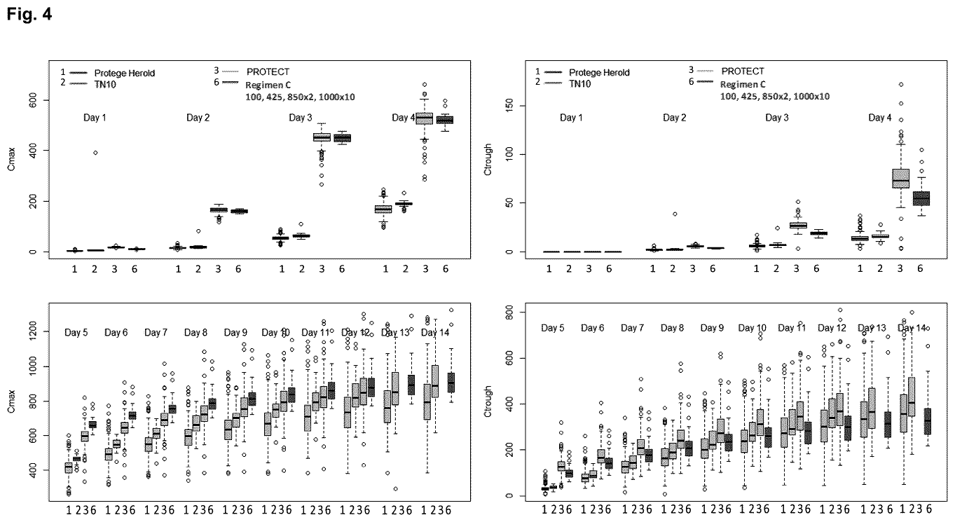

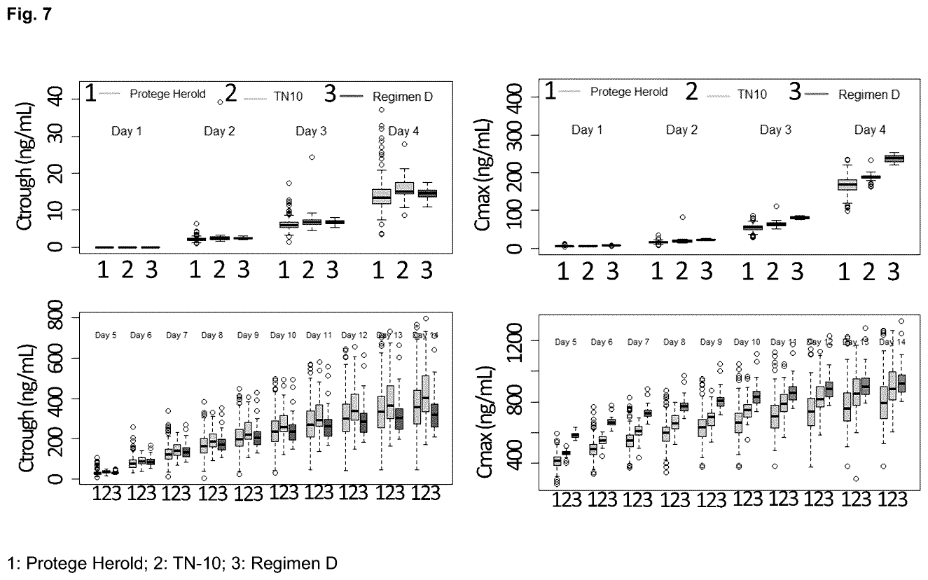

These regimens are predicted not to result in additional safety findings. The predicted Cmax and Ctrough during each day of the 14-day course for the alternative regimens are shown in FIG. 3 (Regimens A and B), FIG. 4 (Regimen C) and FIG. 7 (Regimen D) alongside the values from the historical clinical trials. The Cmax and Ctrough values for Regimens A and B do not exceed those observed in Protégé or TN 10 using the Herold 14-day regimen. The Cmax and Ctrough values for Regimen C do not exceed those observed in the PROTECT 12-day regimen.

Additionally, we evaluated the safety profile of patients in the Protégé and TN-10 studies who have AUC_{inf} greater than and less than or equal to 6000 ng*day/mL. This value represents approximately the geometric mean exposure for the proposed alternative dosing regimens (Table 3). As shown in Table 4 the safety profile based on AEs reported in at least 5% of patients appear comparable between the subset of patients with AUC>6000 ng*day/mL and those with ≤6000 ng*day/mL.

TABLE 3
Simulated AUC_{inf} for Alternative Regimens
for the anti-CD3 antibody

| | AUC_{inf} (ng*day/mL) |
|---|---|
| | Geometric Mean (CV %) |
| Regimen A | 5572 (23%) |
| Regimen B | 5741 (23%) |
| Regimen C | 6297 (24%) |
| Regimen D | 5778 (24%) |

TABLE 4
Safety Profile for Patients with AUC_{inf} > 6000 ng*day/mL and
with ≤6000 ng*day/mL: AEs Reported in ≥5% of Patients

| | | | |
|---|---|---|---|
| | | Protégé/TN-10 | Protégé/TN-10 |
| | | AUC > 6000 | AUC ≤ 6000 |
| System | | ng*day/mL | ng*day/mL |
| Organ | | N = 121 | N = 355 |
| Class | Preferred Term | n (%) | n (%) |

Blood and Lymphatic System Disorders

| | | | | |
|---|---|---|---|---|
| Lymphopenia | 107 | (88.4) | 290 | (81.7) |
| Leukopenia | 89 | (73.6) | 255 | (71.8) |
| Neutropenia | 53 | (43.8) | 157 | (44.2) |
| Thrombocytopenia | 25 | (20.7) | 83 | (23.4) |
| Anaemia | 11 | (9.1) | 43 | (12.1) |
| Eosinophilia | 8 | (6.6) | 23 | (6.5) |

Gastrointestinal Disorders

| | | | | |
|---|---|---|---|---|
| Nausea | 24 | (19.8) | 61 | (17.2) |
| Vomiting | 18 | (14.9) | 42 | (11.8) |
| Diarrhoea | 11 | (9.1) | 23 | (6.5) |
| Dyspepsia | 8 | (6.6) | 6 | (1.7) |
| Abdominal pain upper | 6 | (5.0) | 18 | (5.1) |
| Abdominal pain | 6 | (5.0) | 10 | (2.8) |

General Disorders and Administration Site Conditions

| | | | | |
|---|---|---|---|---|
| Pyrexia | 21 | (17.4) | 82 | (23.1) |
| Fatigue | 16 | (13.2) | 34 | (9.6) |
| Chills | 8 | (6.6) | 32 | (9.0) |

Hepatobiliary Disorders

| | | | | |
|---|---|---|---|---|
| Hyperbilirubinaemia | 13 | (10.7) | 36 | (10.1) |

Immune System Disorders

| | | | | |
|---|---|---|---|---|
| Cytokine release syndrome | 8 | (6.6) | 21 | (5.9) |

Infections and Infestations

| | | | | |
|---|---|---|---|---|
| Upper respiratory tract | 17 | (14.0) | 64 | (18.0) |
| infection | | | | |
| Nasopharyngitis | 17 | (14.0) | 43 | (12.1) |
| Sinusitis | 9 | (7.4) | 20 | (5.6) |
| Influenza | 7 | (5.8) | 10 | (2.8) |
| Rhinitis | 6 | (5.0) | 13 | (3.7) |
| Viral upper respiratory tract | 6 | (5.0) | 5 | (1.4) |
| infection | | | | |
| Pharyngitis | 4 | (3.3) | 21 | (5.9) |

Investigations

| | | | | |
|---|---|---|---|---|
| Haemoglobin decreased | 45 | (37.2) | 99 | (27.9) |
| Alanine aminotransferase | 39 | (32.2) | 106 | (29.9) |
| increased | | | | |
| Blood bicarbonate decreased | 38 | (31.4) | 167 | (47.0) |
| Aspartate aminotransferase | 37 | (30.6) | 118 | (33.2) |
| increased | | | | |
| Blood sodium decreased | 19 | (15.7) | 68 | (19.2) |
| Blood albumin decreased | 16 | (13.2) | 25 | (7.0) |
| Blood calcium decreased | 13 | (10.7) | 42 | (11.8) |
| Blood alkaline phosphatase | 9 | (7.4) | 58 | (16.3) |
| increased | | | | |
| Blood bilirubin increased | 9 | (7.4) | 27 | (7.6) |
| Bilirubin conjugated | 8 | (6.6) | 21 | (5.9) |
| increased | | | | |
| Haematocrit decreased | 8 | (6.6) | 19 | (5.4) |
| Blood phosphorus increased | 8 | (6.6) | 14 | (3.9) |
| Blood potassium decreased | 7 | (5.8) | 24 | (6.8) |
| Blood potassium increased | 7 | (5.8) | 17 | (4.8) |
| Blood phosphorus decreased | 5 | (4.1) | 23 | (6.5) |
| Blood creatinine increased | 4 | (3.3) | 20 | (5.6) |
| Gamma-glutamyltransferase | 4 | (3.3) | 23 | (6.5) |
| increased | | | | |
| Red blood cell count | 3 | (2.5) | 21 | (5.9) |
| decreased | | | | |

Metabolism and Nutrition Disorders

| | | | | |
|---|---|---|---|---|
| Hypocalcaemia | 31 | (25.6) | 77 | (21.7) |
| Hyponatraemia | 24 | (19.8) | 119 | (33.5) |
| Hyperkalaemia | 10 | (8.3) | 45 | (12.7) |
| Hypoalbuminaemia | 9 | (7.4) | 37 | (10.4) |
| Hypokalaemia | 9 | (7.4) | 27 | (7.6) |
| Hypernatraemia | 7 | (5.8) | 14 | (3.9) |
| Hypercalcaemia | 6 | (5.0) | 28 | (7.9) |
| Hypophosphataemia | 4 | (3.3) | 21 | (5.9) |

Nervous System Disorders

| | | | | |
|---|---|---|---|---|
| Headache | 35 | (28.9) | 81 | (22.8) |
| Dizziness | 7 | (5.8) | 12 | (3.4) |

Renal and Urinary Disorders

| | | | | |
|---|---|---|---|---|
| Proteinuria | 21 | (17.4) | 39 | (11.0) |

Respiratory, Thoracic and mediastinal disorders

| | | | | |
|---|---|---|---|---|
| Oropharyngeal pain | 16 | (13.2) | 21 | (5.9) |
| Cough | 15 | (12.4) | 18 | (5.1) |
| Nasal congestion | 9 | (7.4) | 15 | (4.2) |

Skin and Subcutaneous Tissue Disorders

| | | | | |
|---|---|---|---|---|
| Rash | 42 | (34.7) | 129 | (36.3) |
| Pruritus | 11 | (9.1) | 44 | (12.4) |
| Rash papular | 8 | (6.6) | 13 | (3.7) |
| Dry skin | 7 | (5.8) | 5 | (1.4) |
| Rash maculo-papular | 6 | (5.0) | 7 | (2.0) |

Safety information for the precursor product to teplizumab, hOKT3γ1 (Ala-Ala), is available for non-T1D patients, including renal transplant (Woodle 1999), islet transplant (Hering 2004) and psoriatic arthritis (Utset 2002) patients. Total cumulative doses evaluated over a 10 consecutive day treatment period included 76 mg, 46 mg and 40 mg for renal transplant, islet transplant and psoriatic arthritis patients, respectively. The safety events were similar to those observed in T1D patients who received an average total cumulative dose of ˜18-20 mg over a 12-14-day treatment period.

### Example 2: Additional Dosage Regimens

Alternative dosage regimens are shown in the Table 5 below:

TABLE 5
Simulated Alternative 14-days Regimens

| Simulation | | Simulation | |
|---|---|---|---|
| Number | PROTECT-based Regimen^{a} | Number | Herold-based Regimen^{a} |
| 1.1 | 106 - 425 - 850 - 850 - 950 × 10 | 101.1 | 51 - 103 - 207 - 413 - 950 × 10 |
| 1.2 | 106 - 425 - 850 - 950 - 950 × 10 | 101.2 | 51 - 103 - 207 - 413 - 975 × 10 |
| 1.3 | 106 - 425 - 950 - 950 - 950 × 10 | 101.3 | 51 - 103 - 207 - 413 - 1000 × 10 |
| 3.1 | 106 - 425 - 850 - 850 - 970 × 10 | 102.1 | (51 - 103 - 207 - 413 - 826 × 10)*1.20 |
| 3.2 | 106 - 425 - 850 - 970 - 970 × 10 | 102.2 | (51 - 103 - 207 - 413 - 826 × 10)*1.23 |
| 3.3 | 106 - 425 - 970 - 970 - 970 × 10 | 102.3 | (51 - 103 - 207 - 413 - 826 × 10)*1.25 |
| 4.1 | 106 - 425 - 850 - 850 - 990 × 10 | 103.1 | 60 - 125 - 250 - 500 - 1030 × 10 |
| 4.2 | 106 - 425 - 850 - 990 - 990 × 10 | 103.2 | 64 - 129 - 259 - 516 - 1032 × 10 |
| 4.3 | 106 - 425 - 990 - 990 - 990 × 10 | 103.3 | 65 - 130 - 260 - 520 - 1040 × 10 |
| 5.1 | 106 - 425 - 850 - 850 - 940 × 10 | 104.1 | 60 - 125 - 250 - 500 -1000 × 10 |
| 5.2 | 106 - 425 - 850 - 940 - 940 × 10 | 104.2 | 65 - 130 - 260 - 520 -1000 × 10 |
| 5.3 | 106 - 425 - 940 - 940 - 940 × 10 | 104.3 | 65 - 130 - 260 - 600 -1000 × 10 |
| 6.1 | 106 - 425 - 850 - 850 - 945 × 10 | | |
| 6.2 | 106 - 425 - 850 - 945 - 945 × 10 | | |
| 6.3 | 106 - 425 - 945 - 945 - 945 × 10 | | |
| 7.1 | 100 - 425 - 850 - 850 - 1000 × 10 | | |
| 7.2 | 100 - 425 - 850 - 1000 - 1000 × 10 | | |
| 7.3 | 100 - 425 -1000 -1000 - 1000 × 10 | | |
| 8.1 | 100 - 425 - 850 - 850 - 960 × 10 | | |
| 8.2 | 100 - 425 - 850 - 960 - 960 × 10 | | |
| 8.3 | 100 - 425 - 960 - 960 - 960 × 10 | | |

^{a}Doses in μg/m² administered once-a-day as 30 min infusions

### Example 3: Additional Dosage Regimens

Alternative dosage regimen G is shown in the Table 6 below:

TABLE 6

| | Daily Dose (μg/m²) | | | | | |
|---|---|---|---|---|---|---|
| | | | | | | Cumulative dose |
| | | | | | | (μg/m²) [Relative |
| | | | | | | increase from |
| | Day 1 | Day 2 | Day 3 | Day 4 | Days 5-14 | reference] |
| Reference | 51 | 103 | 207 | 413 | 826 | 9,034 [reference] |
| regimen | | | | | | |
| Herold 14- | | | | | | |
| day | | | | | | |

Alternative Regimen

| | | | | | | |
|---|---|---|---|---|---|---|
| Regimen G | 65 | 125 | 250 | 500 | 1070 | 11,640 [29%] |

### Example 4: Dosage and Administration

The teplizumab compositions and methods provided herein are indicated to delay the onset of Stage 3 type 1 diabetes in adults and pediatric patients 8 years of age and older with Stage 2 type 1 diabetes.

Teplizumab-mzwv (also referred herein as Teplizumab) is a CD3-directed monoclonal antibody (humanized IgG1 kappa) that has a molecular weight of approximately 150 kilodalton (kDa) and is expressed from a recombinant Chinese hamster ovary (CHO) cell line.

Teplizumab injection is supplied as a sterile, preservative-free, clear and colorless solution in a 2 mg/2 mL (1 mg/mL) single-dose vial for intravenous use. Each mL contains 1 mg of teplizumab-mzwv, dibasic sodium phosphate (0.26 mg), monobasic sodium phosphate (0.98 mg), polysorbate 80 (0.05 mg), sodium chloride (8.78 mg), and water for injection. The pH is 6.1.

### Clinical Pharmacology

### Mechanism of Action

Teplizumab-mzwv binds to CD3 (a cell surface antigen present on T lymphocytes) and delays the onset of Stage 3 type 1 diabetes in adults and pediatric patients aged 8 years and older with Stage 2 type 1 diabetes. The mechanism may involve partial agonistic signaling and deactivation of pancreatic beta cell auto-reactive T lymphocytes. Teplizumab-mzwv leads to an increase in the proportion of regulatory T cells and of exhausted CD8+ T cells in peripheral blood.

### Pharmacodynamics

Clinical studies have shown that teplizumab-mzwv binds to CD3 molecules on the surface of both CD4+ and CD8+ T cells during treatment, with internalization of the teplizumab-mzwv/CD3 complex from the surface of T cells. Pharmacodynamic effects include lymphopenia in the absence of depletion of T cells with a nadir on the 5th day of dosing, during a 14-day course of teplizumab-mzwv treatment. Teplizumab-mzwv exposure-response relationship and time course of pharmacodynamic response for the safety and effectiveness of teplizumab-mzwv have not been fully characterized.

### Pharmacokinetics

Steady state concentrations of teplizumab-mzwv are not expected to be achieved during the 14-day course of teplizumab.

### Distribution

The central volume of distribution (Vd) of teplizumab-mzwv was 2.27 L in a 60 kg subject.

### Elimination

Teplizumab-mzwv showed saturable binding and elimination. The mean (SD) terminal elimination half-life and clearance of teplizumab-mzwv are 4.5 (0.2) days and 2.7 (0.8) L/day in a 60 kg subject, respectively.

### Metabolism

Teplizumab-mzwv is expected to be metabolized into small peptides by catabolic pathways.

### Specific Populations

No clinically significant differences in the pharmacokinetics of teplizumab-mzwv were observed based on age (8 to 35 years old), biologic sex or racial groups (White, Asians).

### Body Weight

BSA-based dosing normalizes the exposure to teplizumab-mzwv across body weight.

### Immunogenicity

The observed incidence of anti-drug antibodies is highly dependent on the sensitivity and specificity of the assay. Differences in assay methods preclude meaningful comparisons of the incidence of anti-drug antibodies in the studies described below with the incidence of anti-drug antibodies in other studies, including those of teplizumab-mzwv or of other teplizumab products.

In the placebo-controlled study in patients aged 8 years of age and older with Stage 2 type 1 diabetes (Study TN-10), approximately 59% of teplizumab-treated patients developed anti-teplizumab-mzwv antibodies, 46% of whom developed neutralizing antibodies. There is insufficient information to characterize the effects of ADA on pharmacokinetics, pharmacodynamics or effectiveness of teplizumab-mzwv. There was a higher incidence of rash in teplizumab-treated patients who developed anti-teplizumab-mzwv antibodies compared to those who did not develop anti-teplizumab-mzwv antibodies.

### Patient Selection

Select adult patients and pediatric patients 8 years of age and older for TZIELD treatment who have a diagnosis of Stage 2 type 1 diabetes.

Confirm Stage 2 type 1 diabetes by documenting at least two positive pancreatic islet cell autoantibodies (FDA-authorized tests for pancreatic islet cell autoantibodies are recommended) in those who have dysglycemia without overt hyperglycemia. In patients who meet criteria for a diagnosis of Stage 2 type 1 diabetes, ensure the clinical history of the patient does not suggest type 2 diabetes.

### Administration Instructions and Recommended Dosage

Dilute prior to use:
Each vial is intended for single-dose only. Prepare a: Sterile glass vial with 18 mL of 0.9% Sodium Chloride Injection or Polyvinylchloride (PVC) infusion bag with 18 mL of 0.9% Sodium Chloride Injection.
Remove 2 mL of teplizumab from the vial and slowly add to the 18 mL of 0.9% Sodium Chloride Injection. Mix gently by slowly inverting the vial or rocking the infusion bag. The resulting 20 mL diluted solution contains 100 mcg/mL of teplizumab-mzwv.
Using an appropriately sized syringe (e.g., 5 mL), withdraw the volume of diluted solution required on that day for the calculated dose from the 100 mcg/mL solution.
Slowly add contents of the syringe containing teplizumab dose to a 25 mL 0.9% sodium chloride injection PVC infusion bag. Gently rock the infusion bag to ensure that the solution mixes sufficiently. Do not shake.
Discard unused portion of remaining diluted teplizumab solution in the sterile glass vial or PVC infusion bag.
Start the infusion of teplizumab within 2 hours of preparation. If not used immediately, store the diluted solution at room temperature [15° C. to 30° C. (59° F. to 86° F.)] and complete infusion within 4 hours of the start of preparation. Discard the diluted solution if not administered within 4 hours of preparation.

Premedicate prior to infusion for the first 5 days of dosing with oral medications including: (1) a nonsteroidal anti-inflammatory drug (NSAID) or acetaminophen, (2) an antihistamine, and/or (3) an antiemetic. Administer additional doses of premedication if needed.

Administer by intravenous infusion (over a minimum of 30 minutes) once daily for 14 consecutive days.

The recommended dosage for pediatrics and adult patients ages 8 years and older, based on body surface area (BSA) is described in the Table below.

Do not administer two doses on the same day.

TABLE
Recommended Once Daily Dosage in Adults and
Pediatric Patients 8 Years of Age and Older
During the 14-Day Treatment (Regimen D)

| Day(s) | Daily Dosage | |
|---|---|---|
| 1 | 65 | mcg/m² |
| 2 | 125 | mcg/m² |
| 3 | 250 | mcg/m² |
| 4 | 500 | mcg/m² |
| 5 through 14 | 1030 | mcg/m² |

Recommendations Regarding Missed Dose(s)

If a planned infusion is missed, resume dosing by administering all remaining doses on consecutive days to complete the 14-day treatment course.

### Laboratory Evaluation, and Vaccination Prior to Initiation

Prior to initiating, obtain a complete blood count and liver enzyme tests.

Use of is not recommended in patients with:
Lymphocyte count less than 1,000 lymphocytes/mcL
Hemoglobin less than 10 g/dL
Platelet count less than 150,000 platelets/mcL
Absolute neutrophil count less than 1500 neutrophils/mcL
Elevated ALT or AST greater than 2 times the upper limit of normal (ULN) or bilirubin greater than 1.5 times ULN
Laboratory or clinical evidence of acute infection with Epstein-Barr virus (EBV) or cytomegalovirus (CMV)
Active serious infection or chronic active infection other than localized skin infections
Administer all age-appropriate vaccinations prior to starting administration
Administer live-attenuated (live) vaccines at least 8 weeks prior to treatment.
Administer inactivated (killed) vaccines or mRNA vaccines at least 2 weeks prior to treatment.

### Dosage Forms and Strengths

Injection: 2 mg per 2 mL (1 mg/mL) clear and colorless solution in a single-dose vial.

### Contraindications

None.

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

### REFERENCES

1. A. G. Ziegler, M. Rewers, O. Simell, T. Simell, J. Lempainen, A. Steck, C. Winkler, J. Ilonen, R. Veijola, M. Knip, Seroconversion to multiple islet autoantibodies and risk of progression to diabetes in children. Jama 309, 2473-2479 (2013).
2. R. A. Insel, J. L. Dunne, M. A. Atkinson, J. L. Chiang, D. Dabelea, P. A. Gottlieb, C. J. Greenbaum, K. C. Herold, J. P. Krischer, A. Lernmark, R. E. Ratner, M. J. Rewers, D. A. Schatz, J. S. Skyler, J. M. Sosenko, A. G. Ziegler, Staging presymptomatic type 1 diabetes: a scientific statement of JDRF, the Endocrine Society, and the American Diabetes Association. Diabetes Care 38, 1964-1974 (2015).
3. J. P. Krischer, K. F. Lynch, A. Lernmark, W. A. Hagopian, M. J. Rewers, J. X. She, J. Toppari, A. G. Ziegler, B. Akolkar, T. S. Group, Genetic and Environmental Interactions Modify the Risk of Diabetes-Related Autoimmunity by 6 Years of Age: The TEDDY Study. Diabetes Care 40, 1194-1202 (2017).
4. J. L. Mahon, J. M. Sosenko, L. Rafkin-Mervis, H. Krause-Steinrauf, J. M. Lachin, C. Thompson, P. J. Bingley, E. Bonifacio, J. P. Palmer, G. S. Eisenbarth, J. Wolfsdorf, J. S. Skyler, C. TrialNet Natural History, G. Type 1 Diabetes TrialNet Study, The TrialNet Natural History Study of the Development of Type 1 Diabetes: objectives, design, and initial results. Pediatr Diabetes 10, 97-104 (2009).
5. H. T. Siljander, R. Hermann, A. Hekkala, J. Lahde, L. Tanner, P. Keskinen, J. Ilonen, O. Simell, R. Veijola, M. Knip, Insulin secretion and sensitivity in the prediction of type 1 diabetes in children with advanced beta-cell autoimmunity. Eur J Endocrinol 169, 479-503 (2013).
6. N. C. Foster, R. W. Beck, K. M. Miller, M. A. Clements, M. R. Rickels, L. A. DiMeglio, D. M. Maahs, W. V. Tamborlane, R. Bergenstal, E. Smith, B. A. Olson, S. K. Garg, State of Type 1 Diabetes Management and Outcomes from the T1D Exchange in 2016-2018. Diabetes Technol Ther 21, 66-72 (2019).
7. A. Rawshani, N. Sattar, S. Franzen, A. Rawshani, A. T. Hattersley, A. M. Svensson, B. Eliasson, S. Gudbjornsdottir, Excess mortality and cardiovascular disease in young adults with type 1 diabetes in relation to age at onset: a nationwide, register-based cohort study. Lancet 392, 477-486 (2018).
8. S. J. Livingstone, D. Levin, H. C. Looker, R. S. Lindsay, S. H. Wild, N. Joss, G. Leese, P. Leslie, R. J. McCrimmon, W. Metcalfe, J. A. McKnight, A. D. Morris, D. W. Pearson, J. R. Petrie, S. Philip, N. A. Sattar, J. P. Traynor, H. M. Colhoun, g. Scottish Diabetes Research Network epidemiology, R. Scottish Renal, Estimated life expectancy in a Scottish cohort with type 1 diabetes, 2008-2010. JAMA 313, 37-44 (2015).
9. B. Tao, M. Pietropaolo, M. Atkinson, D. Schatz, D. Taylor, Estimating the cost of type 1 diabetes in the U.S.: a propensity score matching method. PLoS One 5, e11501 (2010).
10. C. Evans-Molina, E. K. Sims, L. A. DiMeglio, H. M. Ismail, A. K. Steck, J. P. Palmer, J. P. Krischer, S. Geyer, P. Xu, J. M. Sosenko, G. Type 1 Diabetes TrialNet Study, beta Cell dysfunction exists more than 5 years before type 1 diabetes diagnosis. JCI Insight 3, (2018).
11. E. K. Sims, L. A. DiMeglio, Cause or effect? A review of clinical data demonstrating beta cell dysfunction prior to the clinical onset of type 1 diabetes. Mol Metab 27S, S129-S138 (2019).
12. M. K. Koskinen, O. Helminen, J. Matomäki, S. Aspholm, J. Mykkänen, M. Mäkinen, V. Simell, M. Vähä-Mäkilä, T. Simell, J. Ilonen, Reduced β-cell function in early preclinical type 1 diabetes. European Journal of Endocrinology 174, 251-259 (2016).
13. P. J. Bingley, D. K. Wherrett, A. Shultz, L. E. Rafkin, M. A. Atkinson, C. J. Greenbaum, Type 1 Diabetes TrialNet: A Multifaceted Approach to Bringing Disease-Modifying Therapy to Clinical Use in Type 1 Diabetes. Diabetes Care 41, 653-661 (2018).
14. C. J. Greenbaum, C. Speake, J. Krischer, J. Buckner, P. A. Gottlieb, D. A. Schatz, K. C. Herold, M. A. Atkinson, Strength in Numbers: Opportunities for Enhancing the Development of Effective Treatments for Type 1 Diabetes-The TrialNet Experience. Diabetes 67, 1216-1225 (2018).
15. B. M. Nathan, D. Boulware, S. Geyer, M. A. Atkinson, P. Colman, R. Goland, W. Russell, J. M. Wentworth, D. M. Wilson, C. Evans-Molina, D. Wherrett, J. S. Skyler, A. Moran, J. M. Sosenko, T. Type 1 Diabetes, G. Diabetes Prevention Trial-Type 1 Study, Dysglycemia and Index60 as Prediagnostic End Points for Type 1 Diabetes Prevention Trials. Diabetes Care 40, 1494-1499 (2017).
16. J. M. Sosenko, J. P. Palmer, L. Rafkin-Mervis, J. P. Krischer, D. Cuthbertson, J. Mahon, C. J. Greenbaum, C. C. Cowie, J. S. Skyler, G. Diabetes Prevention Trial-Type 1 Study, Incident dysglycemia and progression to type 1 diabetes among participants in the Diabetes Prevention Trial-Type 1. Diabetes Care 32, 1603-1607 (2009).
17. J. P. Palmer, C-peptide in the natural history of type 1 diabetes. Diabetes Metab Res Rev 25, 325-328 (2009).
18. C. J. Greenbaum, A. M. Anderson, L. M. Dolan, E. J. Mayer-Davis, D. Dabelea, G. Imperatore, S. Marcovina, C. Pihoker, S. S. Group, Preservation of beta-cell function in autoantibody-positive youth with diabetes. Diabetes Care 32, 1839-1844 (2009).
19. W. Hagopian, R. J. Ferry, Jr., N. Sherry, D. Carlin, E. Bonvini, S. Johnson, K. E. Stein, S. Koenig, A. G. Daifotis, K. C. Herold, J. Ludvigsson, I. Protege Trial, Teplizumab preserves C-peptide in recent-onset type 1 diabetes: two-year results from the randomized, placebo-controlled Protege trial. Diabetes 62, 3901-3908 (2013).
20. K. C. Herold, S. E. Gitelman, M. R. Ehlers, P. A. Gottlieb, C. J. Greenbaum, W. Hagopian, K. D. Boyle, L. Keyes-Elstein, S. Aggarwal, D. Phippard, P. H. Sayre, J. McNamara, J. A. Bluestone, A. T. E. S. T. Ab, Teplizumab (anti-CD3 mAb) treatment preserves C-peptide responses in patients with new-onset type 1 diabetes in a randomized controlled trial: metabolic and immunologic features at baseline identify a subgroup of responders. Diabetes 62, 3766-3774 (2013).
21. K. C. Herold, W. Hagopian, J. A. Auger, E. Poumian-Ruiz, L. Taylor, D. Donaldson, S. E. Gitelman, D. M. Harlan, D. Xu, R. A. Zivin, J. A. Bluestone, Anti-CD3 monoclonal antibody in new-onset type 1 diabetes mellitus. N Engl J Med 346, 1692-1698. (2002).
22. B. Keymeulen, E. Vandemeulebroucke, A. G. Ziegler, C. Mathieu, L. Kaufman, G. Hale, F. Gorus, M. Goldman, M. Walter, S. Candon, L. Schandene, L. Crenier, C. De Block, J. M. Seigneurin, P. De Pauw, D. Pierard, I. Weets, P. Rebello, P. Bird, E. Berrie, M. Frewin, H. Waldmann, J. F. Bach, D. Pipeleers, L. Chatenoud, Insulin needs after CD3-antibody therapy in new-onset type 1 diabetes. N Engl J Med 352, 2598-2608 (2005).
23. S. A. Long, J. Thorpe, H. A. DeBerg, V. Gersuk, J. Eddy, K. M. Harris, M. Ehlers, K. C. Herold, G. T. Nepom, P. S. Linsley, Partial exhaustion of CD8 T cells and clinical response to teplizumab in new-onset type 1 diabetes. Sci Immunol 1, (2016).
24. N. Sherry, W. Hagopian, J. Ludvigsson, S. M. Jain, J. Wahlen, R. J. Ferry, Jr., B. Bode, S. Aronoff, C. Holland, D. Carlin, K. L. King, R. L. Wilder, S. Pillemer, E. Bonvini, S. Johnson, K. E. Stein, S. Koenig, K. C. Herold, A. G. Daifotis, I. Protege Trial, Teplizumab for treatment of type 1 diabetes (Protege study): 1-year results from a randomised, placebo-controlled trial. Lancet 378, 487-497 (2011).
25. J. E. Tooley, N. Vudattu, J. Choi, C. Cotsapas, L. Devine, K. Raddassi, M. R. Ehlers, J. G. McNamara, K. M. Harris, S. Kanaparthi, D. Phippard, K. C. Herold, Changes in T-cell subsets identify responders to FcR-nonbinding anti-CD3 mAb (teplizumab) in patients with type 1 diabetes. Eur J Immunol 46, 230-241 (2016).
26. K. C. Herold, B. N. Bundy, S. A. Long, J. A. Bluestone, L. A. DiMeglio, M. J. Dufort, S. E. Gitelman, P. A. Gottlieb, J. P. Krischer, P. S. Linsley, J. B. Marks, W. Moore, A. Moran, H. Rodriguez, W. E. Russell, D. Schatz, J. S. Skyler, E. Tsalikian, D. K. Wherrett, A. G. Ziegler, C. J. Greenbaum, G. Type 1 Diabetes TrialNet Study, An Anti-CD3 Antibody, Teplizumab, in Relatives at Risk for Type 1 Diabetes. N Engl J Med 381, 603-613 (2019).
27. K. Näntö-Salonen, A. Kupila, S. Simell, H. Siljander, T. Salonsaari, A. Hekkala, S. Korhonen, R. Erkkola, J. I. Sipilä, L. Haavisto, Nasal insulin to prevent type 1 diabetes in children with HLA genotypes and autoantibodies conferring increased risk of disease: a double-blind, randomised controlled trial. The Lancet 372, 1746-1755 (2008).
28. G. Diabetes Prevention Trial—Type 1 Diabetes Study, Effects of insulin in relatives of patients with type 1 diabetes mellitus. N Engl J Med 346, 1685-1691 (2002).
29. G. Writing Committee for the Type 1 Diabetes TrialNet Oral Insulin Study, J. P. Krischer, D. A. Schatz, B. Bundy, J. S. Skyler, C. J. Greenbaum, Effect of Oral Insulin on Prevention of Diabetes in Relatives of Patients With Type 1 Diabetes: A Randomized Clinical Trial. JAMA 318, 1891-1902 (2017).
30. H. Elding Larsson, M. Lundgren, B. Jonsdottir, D. Cuthbertson, J. Krischer, A.-I. T. S. G. Di, Safety and efficacy of autoantigen-specific therapy with 2 doses of alum-formulated glutamate decarboxylase in children with multiple islet autoantibodies and risk for type 1 diabetes: A randomized clinical trial. Pediatr Diabetes 19, 410-419 (2018).
31. E. A. Gale, P. J. Bingley, C. L. Emmett, T. Collier, G. European Nicotinamide Diabetes Intervention Trial, European Nicotinamide Diabetes Intervention Trial (ENDIT): a randomised controlled trial of intervention before the onset of type 1 diabetes. Lancet 363, 925-931 (2004).
32. D. L. Eizirik, M. L. Colli, F. Ortis, The role of inflammation in insulitis and beta-cell loss in type 1 diabetes. Nat Rev Endocrinol 5, 219-226 (2009).
33. E. B. Tsai, N. A. Sherry, J. P. Palmer, K. C. Herold, The rise and fall of insulin secretion in type 1 diabetes mellitus. Diabetologia 49, 261-270 (2006).
34. E. Ferrannini, A. Mari, V. Nofrate, J. M. Sosenko, J. S. Skyler, D. P. T. S. Group, Progression to diabetes in relatives of type 1 diabetic patients: mechanisms and mode of onset. Diabetes 59, 679-685 (2010).
35. K. C. Herold, S. Usmani-Brown, T. Ghazi, J. Lebastchi, C. A. Beam, M. D. Bellin, M. Ledizet, J. M. Sosenko, J. P. Krischer, J. P. Palmer, G. Type 1 Diabetes TrialNet Study, beta cell death and dysfunction during type 1 diabetes development in at-risk individuals. J Clin Invest 125, 1163-1173 (2015).
36. N. A. Sherry, E. B. Tsai, K. C. Herold, Natural history of beta-cell function in type 1 diabetes. Diabetes 54 Suppl 2, S32-39 (2005).
37. K. C. Herold, W. Hagopian, J. A. Auger, E. Poumian-Ruiz, L. Taylor, D. Donaldson, S. E. Gitelman, D. M. Harlan, D. Xu, R. A. Zivin, J. A. Bluestone, Anti-CD3 monoclonal antibody in new-onset type 1 diabetes mellitus. N Engl J Med 346, 1692-1698 (2002).
38. K. C. Herold, S. E. Gitelman, U. Masharani, W. Hagopian, B. Bisikirska, D. Donaldson, K. Rother, B. Diamond, D. M. Harlan, J. A. Bluestone, A Single Course of Anti-CD3 Monoclonal Antibody hOKT3{gamma}1(Ala-Ala) Results in Improvement in C-Peptide Responses and Clinical Parameters for at Least 2 Years after Onset of Type 1 Diabetes. Diabetes 54, 1763-1769 (2005).
39. L. M. McLane, M. S. Abdel-Hakeem, E. J. Wherry, CD8 T Cell Exhaustion During Chronic Viral Infection and Cancer. Annu Rev Immunol 37, 457-495 (2019).
40. J. M. Sosenko, J. P. Palmer, C. J. Greenbaum, J. Mahon, C. Cowie, J. P. Krischer, H. P. Chase, N. H. White, B. Buckingham, K. C. Herold, D. Cuthbertson, J. S. Skyler, Patterns of metabolic progression to type 1 diabetes in the Diabetes Prevention Trial-Type 1. Diabetes Care 29, 643-649 (2006).
41. M. M. Bogun, B. N. Bundy, R. S. Goland, C. J. Greenbaum, C-Peptide Levels in Subjects Followed Longitudinally Before and After Type 1 Diabetes Diagnosis in TrialNet. Diabetes Care 43, 1-8 (2020).
42. L. Chatenoud, J. Primo, J. F. Bach, CD3 antibody-induced dominant self tolerance in overtly diabetic NOD mice. J Immunol 158, 2947-2954 (1997).
43. M. B. Davidson, A. L. Peters, D. L. Schriger, An alternative approach to the diagnosis of diabetes with a review of the literature. Diabetes Care 18, 1065-1071 (1995).
44. E. M. Akirav, M. T. Baquero, L. W. Opare-Addo, M. Akirav, E. Galvan, J. A. Kushner, D. L. Rimm, K. C. Herold, Glucose and inflammation control islet vascular density and beta-cell function in NOD mice: control of islet vasculature and vascular endothelial growth factor by glucose. Diabetes 60, 876-883 (2011).
45. N. A. Sherry, J. A. Kushner, M. Glandt, T. Kitamura, A. M. Brillantes, K. C. Herold, Effects of autoimmunity and immune therapy on beta-cell turnover in type 1 diabetes. Diabetes 55, 3238-3245 (2006).
46. A. L. Perdigoto, P. Preston-Hurlburt, P. Clark, S. A. Long, P. S. Linsley, K. M. Harris, S. E. Gitelman, C. J. Greenbaum, P. A. Gottlieb, W. Hagopian, A. Woodwyk, J. Dziura, K. C. Herold, N. Immune Tolerance, Treatment of type 1 diabetes with teplizumab: clinical and immunological follow-up after 7 years from diagnosis. Diabetologia 62, 655-664 (2019).
47. K. C. Herold, S. L. Bucktrout, X. Wang, B. W. Bode, S. E. Gitelman, P. A. Gottlieb, J. Hughes, T. Joh, J. B. McGill, J. H. Pettus, S. Potluri, D. Schatz, M. Shannon, C. Udata, G. Wong, M. Levisetti, B. J. Ganguly, P. D. Garzone, R. N. W. Group, Immunomodulatory activity of humanized anti-IL-7R monoclonal antibody RN168 in subjects with type 1 diabetes. JCI Insight 4, (2019).
48. M. Battaglia, M. S. Anderson, J. H. Buckner, S. M. Geyer, P. A. Gottlieb, T. W. H. Kay, A. Lernmark, S. Muller, A. Pugliese, B. O. Roep, C. J. Greenbaum, M. Peakman, Understanding and preventing type 1 diabetes through the unique working model of TrialNet. Diabetologia 60, 2139-2147 (2017).
49. L. Yu, D. C. Boulware, C. A. Beam, J. C. Hutton, J. M. Wenzlau, C. J. Greenbaum, P. J. Bingley, J. P. Krischer, J. M. Sosenko, J. S. Skyler, G. S. Eisenbarth, J. L. Mahon, G. Type 1 Diabetes TrialNet Study, Zinc transporter-8 autoantibodies improve prediction of type 1 diabetes in relatives positive for the standard biochemical autoantibodies. Diabetes Care 35, 1213-1218 (2012).
50. A. American Diabetes, 2. Classification and Diagnosis of Diabetes: Standards of Medical Care in Diabetes 2019. Diabetes Care 42, S13-S28 (2019).
51. C. Steele, W. A. Hagopian, S. Gitelman, U. Masharani, M. Cavaghan, K. I. Rother, D. Donaldson, D. M. Harlan, J. Bluestone, K. C. Herold, Insulin Secretion in Type 1 Diabetes. Diabetes 53, 426-433 (2004).
52. K. S. Polonsky, J. Licinio-Paixao, B. D. Given, W. Pugh, P. Rue, J. Galloway, T. Karrison, B. Frank, Use of biosynthetic human C-peptide in the measurement of insulin secretion rates in normal volunteers and type I diabetic patients. J Clin Invest 77, 98-105 (1986).
53. E. Van Cauter, F. Mestrez, J. Stuns, K. S. Polonsky, Estimation of insulin secretion rates from C-peptide levels. Comparison of individual and standard kinetic parameters for C-peptide clearance. Diabetes 41, 368-377 (1992).
54. S. A. Long, J. Thorpe, K. C. Herold, M. Ehlers, S. Sanda, N. Lim, P. S. Linsley, G. T. Nepom, K. M. Harris, Remodeling T cell compartments during anti-CD3 immunotherapy of type 1 diabetes. Cell Immunol 319, 3-9 (2017).
55. Besser R. E. J., et al. “ISPAD clinical practice consensus guidelines 2022: Stages of type 1 diabetes in children and adolescents” Pediatr. Diabetes 2022: 1-13
56. Classification and Diagnosis of Diabetes: Standard of Medical Care in Diabetes-2022. Diabetes Care 2022; 45 (Suppl. 1): S17-S38

1. A method of preventing or delaying onset of clinical type 1 diabetes (T1D), comprising:
administering a prophylactically effective amount of an anti-CD3 antibody to a non-diabetic subject who is at risk of T1D,
wherein the prophylactically effective amount has a cumulative dose of from about 10,500 μg/m² to about 14,000 μg/m².

2. The method of claim 1, wherein the subject is 8 years of age or older.

3. The method of claim 1, wherein the subject is 5 years of age or older.

4. The method of claim 1, wherein the subject is 3 years of age or older.

5. The method of claim 1, wherein the subject is 1 year of age or older.

6. The method of claim 1, wherein the subject is 1 year old or younger.

7. The method of claim 1, wherein the non-diabetic subject is a relative of a patient with T1D.

8. The method of claim 1, further comprising determining that the non-diabetic subject (1) is negative for zinc transporter 8 (ZnT8) antibodies, (2) is HLA-DR4+, and/or (3) is not HLA-DR3+.

9. The method of claim 1, wherein the non-diabetic subject has 2 or more diabetes-related autoantibodies selected from islet cell antibodies (ICA), insulin autoantibodies (IAA), and antibodies to glutamic acid decarboxylase (GAD), tyrosine phosphatase (IA-2/ICA512) or ZnT8.

10. The method of claim 1, wherein the non-diabetic subject has abnormal glucose tolerance on oral glucose tolerance test (OGTT).

11. The method of claim 10, wherein the abnormal glucose tolerance on OGTT is a fasting glucose level of 110-125 mg/dL, or 2 hour plasma of ≥140 and <200 mg/dL, or an intervening glucose value at 30, 60, or 90 minutes on OGTT>200 mg/dL.

12. The method of claim 1, wherein the non-diabetic subject does not have antibodies against ZnT8.

13. The method of claim 1, wherein the non-diabetic subject is HLA-DR4+ and is not HLA-DR3+.

14. The method of claim 1, wherein the anti-CD3 antibody is selected from teplizumab, otelixizumab or foralumab.

15. The method of claim 1, wherein the anti-CD3 antibody is teplizumab.

16. The method of claim 14, wherein the prophylactically effective amount comprises a 14-day course of subcutaneous (SC) injection or intravenous (IV) infusion or oral administration of the anti-CD3 antibody with a cumulative dose of from about 11,000 μg/m² to about 14,000 μg/m².

17. The method of claim 15, comprising administering a 14-day course IV infusion at about 60 μg/m² on day 1, about 125 μg/m² on day 2, about 250 μg/m² on day 3, and about 500 μg/m² on day 4, and a dose of about 1,000 μg/m² on each of days 5-14.

18. The method of claim 15, comprising administering a 14-day course IV infusion at about 60 μg/m², about 125 μg/m² on day 1, about 250 μg/m² on day 2, and about 500 μg/m² on day 3, and a dose of about 1,030 μg/m² on each of days 5-14.

19. The method of claim 15, comprising administering a 14-day course IV infusion at about 100 μg/m² on day 1, about 425 μg/m² on day 2, about 850 μg/m² on day 3, about 850 μg/m² on day 4, and a dose of about 1,000 μg/m² on each of days 5-14.

20. The method of claim 15, comprising administering a 14-day course IV infusion at about 65 μg/m² on day 1, about 125 μg/m² on day 2, about 250 μg/m² on day 3, and about 500 μg/m² on day 4, and a dose of about 1,070 μg/m² on each of days 5-14.

21. The method of claim 15, comprising administering a 14-day course IV infusion at about 65 μg/m² on day 1, about 125 μg/m² on day 2, about 250 μg/m² on day 3, and about 500 μg/m² on day 4, and a dose of about 1,030 μg/m² on each of days 5-14.

22. The method of claim 15, wherein the prophylactically effective amount delays median time to clinical diagnosis of T1D by at least 50%, at least 80%, or at least 90%, or at least 12 months, at least 18 months, at least 24 months, at least 36 months, at least 48 months, or at least 60 months.

23. The method of claim 1, further comprising determining, prior to or after the administering step, that the non-diabetic subject has more than about 10% TIGIT+KLRG1+CD8+ T-cells in all CD3+ T cells, which is indicative of successful prevention or delay of the onset of clinical T1D.

24. The method of claim 23, wherein the determining of TIGIT+KLRG1+CD8+ T-cells is by flow cytometry.

25. The method of claim 1, further comprising determining a decrease in a percentage of CD8+ T cells expressing proliferation markers Ki67 and/or CD57.

26. A method of preventing or delaying onset of clinical type 1 diabetes (T1D), comprising:
administering to a non-diabetic subject 8 years of age or older who is at risk of T1D a 14-day course IV infusion of teplizumab at about 65 μg/m² on day 1, about 125 μg/m² on day 2, about 250 μg/m² on day 3, and about 500 μg/m² on day 4, and a dose of about 1,030 μg/m² on each of days 5-14.

27. A method of delaying onset of Stage 3 type 1 diabetes (T1D), comprising:
administering to a subject in need thereof who has a diagnosis of stage 2 T1D a 14-day course IV infusion of teplizumab at about 65 μg/m² on day 1, about 125 μg/m² on day 2, about 250 μg/m² on day 3, and about 500 μg/m² on day 4, and a dose of about 1,030 μg/m² on each of days 5-14.

28. The method of claim 27, wherein the subject in need thereof is an adult.

29. The method of claim 27, wherein the subject in need thereof is a pediatric subject 8 years of age or older.

30. The method of claim 27, wherein the subject in need thereof is a pediatric subject 5 years of age or older.

31. The method of claim 27, wherein the subject in need thereof is a pediatric subject 3 years of age or older.

32. The method of claim 27, wherein the subject in need thereof is a pediatric subject 1 year of age or older.

33. The method of claim 27, wherein the subject in need thereof is a pediatric subject 1 year old or younger.

34. The method of claim 27, the method comprising documenting two or more positive pancreatic islet autoantibodies in the subject who has dysglycemia without overt hyperglycemia before administering the 14-day course.

35. The method of claim 27, wherein the subject in need thereof has dysglycemia without overt hyperglycemia and has two or more pancreatic islet autoantibodies.

36. The method of claim 35, wherein the two or more pancreatic islet autoantibodies comprise islet cell antibodies (ICA), insulin autoantibodies (IAA), and antibodies to glutamic acid decarboxylase (GAD), tyrosine phosphatase (IA-2/ICA512) or ZnT8.

37. The method of claim 27, the method comprising administering on at least each of days 1-5, and prior to the administering of the 14-day course IV infusion, an effective amount of a nonsteroidal anti-inflammatory drug (NSAID), acetaminophen, an antihistamine, an antiemetic or a combination thereof.

38. The method of claim 37, the method comprising administering orally the NSAID, acetaminophen, antihistamine, antiemetic or combination thereof.

39. The method of claim 27, the method comprising administering the 14-day course IV infusion once daily for 14 consecutive days over a period of at least 30 minutes.

40. A method of prognosing responsiveness of an anti-CD3 antibody in preventing or delaying onset of type 1 diabetes (T1D), the method comprising:
administering a prophylactically effective amount of an anti-CD3 antibody to a non-diabetic subject who is at risk of T1D, wherein the prophylactically effective amount has a cumulative dose of about 10,500 μg/m² to about 14,000 μg/m²; and
determining C-peptide area under the curve (AUC): glucose AUC ratio, wherein an increase in the ratio indicates responsiveness to the anti-CD3 antibody and/or non-progression to clinical T1D.

41.-42. (canceled)


SEQUENCE LISTING (2 sequences)

SEQ ID NO: 1 - protein, 213 aa, unidentified
  REGION 1..213: note = Synthetic
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, unidentified
  REGION 1..449: note = Synthetic
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK